(19) United States
(12) Patent Application Publication
(10) Pub. No.: US 2026/0120835 A1
(43) Pub. Date: Apr. 30, 2026

(54) POINT OF CARE SYSTEMS AND METHODS FOR PRESENTING A PATIENT-SPECIFIC, DISEASE SPECIFIC EVIDENCE-BASED COST OPTIMIZED PATH OF CARE

(71) Applicant: Outcomes Matter Innovations LLC, Jersey City, NJ (US)

(72) Inventors: Andrew L. Pecora, Rumson, NY (US); Casey Simon, Mahwah, NJ (US); Stephen Pecora, Rumson, NJ (US)

(73) Assignee: Outcomes Matter Innovations LLC, Jersey City, NJ (US)

(21) Appl. No.: 19/047,137

(22) Filed: Feb. 6, 2025

Related U.S. Application Data
(60) Provisional application No. 63/670,405, filed on Jul. 12, 2024.

(51) Int. Cl. G16H 20/00; G16H 10/60; G16H 70/00

(52) U.S. Cl. CPC G16H 20/00; G16H 10/60; G16H 70/00

(57) ABSTRACT

The present disclosure provides systems and methods that establish in real time at a point of care a patient-specific and evidence-based cost optimized path of care while maintaining expected clinical outcomes for a patient with a disease.

### CROSS-REFERENCE TO RELATED APPLICATIONS

The present application claims the benefit of priority to U.S. provisional application 63/670,405 (filed 12 Jul. 2024), the contents of which are incorporated herein by reference.

### FIELD OF THE INVENTION

The described invention relates to point of care systems and methods for presenting a patient-specific, disease specific, evidence-based cost-optimized path of care in order to maintain or improve the expected relevant clinical outcome and reduce the total cost of care.

### BACKGROUND OF THE INVENTION

The United States expends more per capita on healthcare than any other nation and clinical outcomes are not demonstrably better. In fact, it is well accepted that up to one quarter of the annual $4 trillion-dollar US healthcare expenditure is wasted because it does not contribute to either greater access or better outcomes. Specialty care, meaning healthcare services provided by specialists with advanced training and expertise in specific areas of medicine, including cardiovascular disease, neurological disorders, or cancer, where the majority of healthcare expenditure occurs, has extensive care variation in seemingly similar patient types (See Pecora, AL., et al. JCO Clinical Cancer Informatics (2018) https://doi.org/10.1200/CC1.18/00006) leading to significant cost variation and waste without better clinical outcomes.

A National Academy of Medicine committee found in 2013 that adult life expectancy in the U.S. has fallen below that of 56 countries on six continents. Although low-income and minority Americans bear the heaviest burden, even well-off Americans with a college degree and health insurance tend to be sicker and to die sooner than their counterparts in other countries. [U.S. Health In International Perspective: Shorter Lives, Poorer Health. Woolf, S H and Aron, L. Editors. (2013) The National Academies Press, Washington DC].

In 2021, the Commonwealth Fund analyzed the performance of America's health system compared to those of other high-income countries [https://www.commonwealthfund.org/publications/fund-reports/2021/aug/mirror-mirror-2021-reflecting-poorly, accessed Apr. 15, 2024].

71 performance measures were analyzed across five domains—access to care, care process, administrative efficiency, equity, and health care outcomes—drawn from Commonwealth Fund international surveys conducted in each country and administrative data from the Organization for Economic Co-operation and Development and the World Health Organization.

The 2021 Commonwealth Fund Report showed that top-performing countries overall are Norway, the Netherlands, and Australia. The United States ranks last among 11 nations overall, despite spending far more of its gross domestic product on health care. The U.S. ranks last on access to care, administrative efficiency, equity, and health care outcomes, but second on measures of care process, which includes measures of preventive care, safe care, coordinated care and engagement and patient preferences. [Id.]

The U.S. has the poorest performance on the affordability subdomain, scoring much lower than even the next-lowest country, Switzerland. Compared to residents of the U.S., residents of the Netherlands, the United Kingdom, Norway, and Germany are much less likely to report that their insurance denied payment of a claim or paid less than expected. [Id.]

U.S. doctors are the most likely to have trouble getting their patients medication or treatment because of restrictions on insurance coverage. Compared to most of the other countries, larger percentages of adults in the U.S. say they spend a lot of time on paperwork related to medical bills. For nonemergency care, U.S. and Canadian adults are also more likely to visit the emergency department—a less efficient option than seeing a regular doctor.

Four features distinguish top performing countries from the United States: 1) they provide for universal coverage and remove cost barriers; 2) they invest in primary care systems to ensure that high-value services are equitably available in all communities to all people; 3) they reduce administrative burdens that divert time, efforts, and spending from health improvement efforts; and 4) they invest in social services, especially for children and working-age adults. [Id.]

Nearly one-third of pharmaceutical spending in the United States is for clinician-administered drugs (e.g., infusions). [Feldman, W B., et al. JAMA Intern. Med. (2022) 182 (1): 83-86, citing Pew Charitable Trust. A look at drug spending in the US: estimates and projections from various stakeholders. Published online Feb. 27, 2018. Accessed Jul. 11, 2021. https://www.newtrusts.org/en/research-and-analysis/fact-sheets/2018/02/a-look-at-drug-spending-in-the-us]. Medicare Part B reimbursement for these drugs is set at the average sale price (ASP) plus a 6% markup (or 4.3% during budget sequestration) [Id., citing Centers for Medicare & Medicaid Services, Medicare Part B drug spending dashboard, Accessed Jul. 11, 2021. https://www.cms.gov/Research-Statistics-Data-and-Systems/Statistics-Trends-and-Report/Information-on-Prescription-Drugs/MedicarePartB]. In contrast, hospitals and physician offices charge commercial insurers whatever price they negotiate, and they retain any difference between the negotiated price and cost of acquisition. A transparency rule that took effect on Jan. 1, 2021, required hospitals to post payer-specific negotiated prices for all items and services, including clinician-administered drugs.

The Inflation Reduction Act of 2022 has been touted as expanding benefits, lowering drug costs, and improving access to affordable treatments. [Fact sheet, CMS, Medicare Drug Price Negotiation Program: Manufacturer Agreements for Selected Drugs for Initial Price Applicability Year 2026. https://www.cms.gov/files/document/fact-sheet-manufacturer-agreements-selected-drugs-ipay-2026.pdf; Accessed Apr. 11, 2024].

With the passage of the Inflation Reduction Act, beginning in 2026, the Centers for Medicare and Medicaid Services (CMS) will be able to negotiate the price of certain high expenditure, single source drugs without generic or biosimilar competition with pharmaceutical companies. [Jaksa, A. et al. J. Comparative Effectiveness Research (2023) e231025]. The U.S. government subsequently selected the first ten drugs that will be subject to the CMS price negotiations: apixaban (Eliquis®); empagliflozin (Jardiance®); riaroxaban (Xarelto®); sitagliptin (Januvia®); dapagliflozin (Farxiga®); sacubitril/valsartan (Entresto®); etanercept (Enbrel®); ibrutinib (Imbruvica®); usckinumab (Stelera®); insulin.aspart (NovoLog®, etc.). As part of the drug pricing negotiation process, CMS will be evaluating therapeutic advances as well as the comparative effectiveness of a given drug compared with its therapeutic alternatives in the Medicare patient population and the extent to which the drug addresses unmet needs. CMS will consider evidence related to therapeutic alternatives as well as other factors, such as costs of research and development and production and distribution of the selected drug. These evaluations will largely rely on published comparative studies, of which real world evidence investigations will likely play a crucial role.

However, the reported analysis found that the evidence-base for these 10 drugs is limited. Of the 170 comparative real-world evidence (RWE) studies (which is one component of the clinical evidence that CMS will consider) identified in the literature, only about a third (32.4%) were conducted using Medicare data. Additionally, the number of studies varied considerably by drug, with the factor Xa inhibitors apixaban (Eliquis®) and rivaroxaban (Xarelto®) (drug class anticoagulant; cardiovascular) studies accounting for the majority (67.1%). The identified studies also examined a wide range of indications, outcomes, and drug comparators.

A new approach is desperately needed to address the inadequacies of the U.S. healthcare system and put it on a cost-optimized evidence-based path.

The described method for identifying and presenting at the point of care and in real time a patient and disease specific cost-optimized evidence-based path of care enables a physician to choose a course of care resulting in a lower total cost of care while maintaining or improving the relevant expected clinical outcome The method, which eliminates the need for prior authorization and prior (pre) certifications, provides access to both lower cost therapeutic advances and to effective treatments for all patients, including underserved populations whose insurance is inadequate for coping with a catastrophic illness.

Although described with respect to cancer conditions, the described method can be used for any clinical condition (e.g., cardiovascular disease, metabolic disease (diabetes), immune mediated diseases (e.g., autoimmune disease, rheumatology), organ transplantation; neurologic and neurodegenerative disorders; renal and bladder disorders, musculoskeletal disorders, pulmonary diseases, hematologic disorders, cutaneous disorders, infectious diseases, digestive and hepatic disorders), behavioral health disorders and disorders of child birth.

### SUMMARY OF THE INVENTION

According to one aspect, the present disclosure provides a method for identifying and presenting at a point of care a care-optimized evidence-based path of care for a patient with a disease, comprising: (1) determining treatment choices based on evidence-based paths of care deemed appropriate for the patient by: (a) accessing a plug-in comprising an interface that accepts a patient specific disease-specific characterization code (PSDSCC); (b) by communication with an action code engine translating the PSDSCC into an action code; (c) the action code collecting a set of action code-associated switches via a user interface, wherein the action code links to actions by specifying a set of associated switches; (d) generating, from values of the action code and the set of associated switches, a set of clinically equivalent candidate generic treatment regimens comprising interchangeable regimens for patients that exhibit the disease-specific characterization elements presented, wherein drugs are identified only by class; (2) optimizing, by communication with a care-optimization engine, costs of all aspects of care (including site of service, drug, drug regimen, surgical procedures, radiation therapy, lab tests, imaging, emergency room visits, in-patient hospitalization, discharge to home, and end of life care) that are changeable in the candidate generic treatment regimens to care for a specific illness by: (a) identifying, characterizing, cost-assessing, and either avoiding or reducing its cost, and (b) returning an evidence-based actual treatment regimen comprising a most effective treatment plan; (3) presenting on a display device at the point of care at a time of treatment decision by a user: (i) an evidence-based actual treatment regimen comprising the evidenced path of care associated with a best expected clinical outcome and a reduced total cost of care; and (ii) an ideal value-based path of care; (4) collecting, analyzing and presenting by display economic outcome data and clinical outcome data for monitoring and reporting the data over time; and (5) optimizing drug utilization in accordance with site-specific purchasing contracts that include economic benefits comprising acquisition cost rebates to enhance practice economic margins and reduce payor cost through reducing total cost of care and delivering an expected relevant clinical outcome.

In some embodiments of the method, the economic benefits include lower drug acquisition cost and lower administrative cost. In some embodiments, lower administrative cost occurs through removal of precertification and prior authorization. In some embodiments, the method is performed in real-time. In some embodiments, the PSDSCC is a derived expression comprising elements collected via a user interface; or the PSDSCC is a derived expression comprising a punctuated string comprising letters and symbols (OMIC.); or the PSDSCC is an expression derived from alphabet letters, symbols and/or numbers of a common language and include Greek, Cyrillic and/or Latin alphabet, symbols, and/or numbers; or the PSDSCC is an ICD code; or the PSDSCC patient-specific disease-specific classification code is a derived expression comprising an avatar; or the PSDSCC is a derived expression comprising biometric patterns; or the PSDSCC is a derived expression comprising sound patterns; or the PSDSCC is a derived expression comprising electromagnetic wave patterns. In some embodiments, the electromagnetic wave patterns comprise patterns of light; and the patterns of light include patterns of nonconvergent visible light, patterns of convergent laser light, patterns of photons of light, or a combination thereof.

In some embodiments, the PSDSCC is an expression derived by machine learning or by deep learning.

In some embodiments, each PSDSCC is associated with one action code, but each action code comprises a plurality of PSDSCC s; and the action code resolves a choice of values of a plurality of disease elements.

In some embodiments, the associated switches specified by each action code comprise optional elements valued as true/false, positive/negative or present/absent.

In some embodiments, the components of care in step (2) include a pharmacy formulation component; a drug dispensing component, a supportive care component; a laboratory test component, an imaging component; a hospital or emergency room visit component; a treatment component, a surgery component, a radiation component; and a psychotherapy/counseling component.

In some embodiments, the presenting is by a graphical user interface (GUI).

In some embodiments, the economic outcome comprises one or more of: lowering the total cost of care; generating shared savings; eliminating prior authorization and precertification; or providing a basis for the negotiation of drug price by the healthcare provider's business side in order to optimize average sale price while maximizing rebates.

In some embodiments, the economic outcome comprises equalizing cost of acquisition of drug to reimbursement plus rebate.

In some embodiments, the clinical outcome is monitoring progression-free, disease-free survival and overall survival for any treatment regimen selected; or monitoring delivered dose intensity (DDI) and Pathologic Complete Response (CR) for patients where a pathway is chosen by a provider; or a combination thereof.

In some embodiments, the disease is a cancer. In some embodiments, the cancer is an AIDS-related cancer; a breast cancer, a digestive/gastrointestinal cancer; an endocrine and neuroendocrine cancer; an eye cancer; a genitourinary cancer; a germ cell cancer; a gynecologic cancer; a head and neck cancer; a hematologic/blood cancer; a musculoskeletal cancer; a neurologic cancer; a respiratory/thoracic cancer; a skin cancer; or an unknown primary cancer. In some embodiments, the cancer is a bladder cancer, breast cancer, colon and rectal cancer; endometrial cancer; kidney cancer; leukemia; liver cancer; lung cancer; melanoma; non-Hodgkin lymphoma; pancreatic cancer; prostate cancer; or thyroid cancer. In some embodiments, in step 1(c), the set of clinically equivalent candidate generic treatment regimens comprising interchangeable regimens comprises NCCN-approved regimens for patients that exhibit the disease-specific characterization elements presented.

In some embodiments, the economic outcome comprises establishing a rule for distribution of shared savings such that shared savings only occur for those patients with cancer where the optimal pathway is chosen and delivered dose intensity (DDI) is greater than or equal to 80%.

According to another aspect the present disclosure provides a non-transitory computer-readable medium storing instructions that, when executed by one or more processors of a computing system, perform the method of any one of claims 1 to 19.

According to another aspect, the present disclosure provides a system for facilitating identification and presentation at a point of care of a cost-optimized evidence based path of care for a patient with a disease comprises storage and one or more processors in communication with the storage and configured to execute instructions from the storage that, when executed by one or more processors of a computing system, cause the system upon an input disease element accessing a plug-in comprising an interface that accepts a patient-specific disease-specific characterization code (PSDSCC) to: (1) determine treatment choices based on evidence-based paths of care deemed appropriate for the patient by: (a) by communication with an action code engine translate the PSDSCC into an action code, and collect a set of action code-associated switches; (b) generate from values of the action code and the set of associated switches a set of clinically equivalent candidate generic treatment regimens comprising interchangeable regimens for patients that exhibit the disease-specific characterization elements presented, wherein drugs are identified only by drug class; (2) optimize, by communication with a care-optimization engine costs of all aspects of care in the generic treatment regimens that are changeable to care for a specific illness by a) identifying, characterizing, cost-assessing, and either avoiding or reducing their cost, and b) return an actual treatment regimen comprising a most effective treatment plan; (3) present on a display device at the point of care at a time of treatment decision by a user: (i) the evidence-based actual treatment regimen comprising the evidenced path of care associated with a best expected clinical outcome and a ranked total cost of care and information regarding the paths of care; and (ii) an ideal value-based path of care; (4) collect, analyze and present by display economic outcome data and clinical outcome data for monitoring and reporting of the data over time; and (5) optimize drug utilization in accordance with site-specific purchasing contracts that include acquisition cost rebates and other economic benefits to enhance practice economic margins and reduce payor cost through reducing total cost of care and delivering an expected relevant clinical outcome.

In some embodiments of the system, the other economic benefits include lower drug acquisition cost and lower administrative cost.

In some embodiments lower administrative cost is through removal of precertification and prior authorization.

In some embodiments the steps are performed in real-time.

In some embodiments, the PSDSCC is a derived expression comprising elements collected through a user interface; or the PSDSCC is a derived expression comprising a punctuated string comprising letters and symbols (OMIC); or the PSDSCC is an ICD code; or the PSDSCC is a derived expression comprising an avatar; or the PSDSCC is a derived expression comprising biometric patterns; or the PSDSCC is a derived expression comprising sound patterns; or the PSDSCC is a derived expression comprising electromagnetic wave patterns. In some embodiments, the electromagnetic wave patterns comprise patterns of light; and the patterns of light include patterns of nonconvergent visible light, patterns of convergent laser light, patterns of photons of light, or a combination thereof.

In some embodiments, the PSDSCC is an expression derived by machine learning or by deep learning.

In some embodiments, each PSDSCC is associated with one action code, but each action code comprises a plurality of PSDSCCs; and the action code resolves a choice of values of a plurality of disease elements.

In some embodiments, the associated switches specified by each action code comprise optional elements valued as true/false, positive/negative or present/absent

In some embodiments, the presenting by display is by a graphical user interface (GUI).

In some embodiments, the components of care in step (3) include a pharmacy formulation component; a drug dispensing component, a supportive care component; a laboratory test component, an imaging component; a hospital visit component; a treatment component, a surgery component, a radiation component; and a psychotherapy/counseling component.

In some embodiments, economic outcomes comprise one or more of: lowering the total cost of care; or generating shared savings; or eliminating prior authorization; or providing a basis for the negotiation of drug price by the healthcare provider's business side in order to optimize average sale price while maximizing rebates.

In some embodiments, the economic outcome comprises equalizing cost of acquisition of drug to reimbursement plus rebate.

In some embodiments, the clinical outcome is one or more of: monitoring progression-free, disease-free survival and overall survival for any treatment regimen selected; or monitoring delivered dose intensity (DDI) and Pathologic Complete Response (CR) for patients where a pathway is chosen by a healthcare provider.

The system of claim 21, wherein the disease is a cancer. In some embodiments, the cancer is an AIDS-related cancer; a breast cancer, a digestive/gastrointestinal cancer; an endocrine and neuroendocrine cancer; an eye cancer; a genitourinary cancer; a germ cell cancer; a gynecologic cancer; a head and neck cancer; a hematologic/blood cancer; a musculoskeletal cancer; a neurologic cancer; a respiratory/thoracic cancer; a skin cancer; or an unknown primary cancer. In some embodiments, the cancer is a bladder cancer, breast cancer, colon and rectal cancer; endometrial cancer; kidney cancer; leukemia; liver cancer; lung cancer; melanoma; non-Hodgkin lymphoma; pancreatic cancer; prostate cancer; or thyroid cancer. In some embodiments, in step 1(c), the set of clinically equivalent candidate generic treatment regimens comprising interchangeable regimens comprises NCCN-approved regimens for patients that exhibit the disease-specific characterization elements presented. In some embodiments, the economic outcome comprises establishing a rule for distribution of shared savings such that shared savings only occur for those patients with cancer where the optimal pathway is chosen and delivered dose intensity (DDI) is greater than or equal to 80%.

### BRIEF DESCRIPTION OF THE DRAWINGS

FIG. 1A is a block diagram depicting exemplary plugins for a method 100 for identifying and presenting at a point of care a cost-optimized evidence-based path of care.

FIG. 1B is a block diagram depicting steps of the method whereby the patient disease-specific characterization code is translated by an action code engine [0120] into an action code, which collects a set of associated switches via a user interface (UI). The result of this collecting is a set of clinically equivalent candidate generic treatment regimens [0130], wherein drugs are listed by class. The generic regimens and action code engine communicate with a care-optimization engine to create regimens with optimal drugs [0150]. Treatment selections are selected via a UI to generate a chosen regimen with optimal drugs [0160]. Possible substitutions with equivalent drugs [0170] are collected from a healthcare provider via a UI. A treatment other than those indicated may be selected. In this case, a justification is required in order for the patient to remain in a value-based care program. The possible justifications are represented by “Justification Switches” and include reasons, such as ECOG Performance Status ≥3, Significant Comorbidity, Adverse Social Determinants of Health, and Patient Choice [180]. The output result is a final actual treatment regimen comprising a most cost-effective treatment plan.

FIG. 2A and FIG. 2B are screenshots showing a graphical user interface (GUI) for transmitting and displaying information in accordance with inputs for an embodiment for a patient with breast cancer. FIG. 2A shows the Required and Dependent Elements. FIG. 2B shows the OMIC, Action Code, Switches and Treatments.

FIG. 3 schematically depicts a network 300, alternately described as a networked computing system, for implementing some aspects in accordance with some embodiments. Network 300 may include at least one computing system 305, at least one client device 315, and data storage 310 that may be in the form of one or more databases. In some embodiments, computing system 305, client device 315, readout device 317, and/or data storage 310 may be connected to network 320. However, in other embodiments, two or more of computing system 305, client device 315, and/or data storage 310 may be connected directly with each other, without network 320.

FIG. 4 schematically depicts inputs from a client device interfacing with a communications network and network architecture for a computer system as described in one embodiment.

### DESCRIPTION OF THE INVENTION

### Definitions

Throughout the specification and claims of the present disclosure, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

As used herein, the term “about” means plus or minus 10% of the numerical value of the number with which it is being used. Therefore, about 50% means in the range of 40%-60%.

The term “acinic cell carcinoma of the breast” as used herein refers to a rare special subtype of breast cancer in the category of salivary gland-type tumors. It is morphologically similar to acinic cell carcinomas of salivary glands and pancreas and has a triple-negative phenotype (estrogen receptor-negative, progesterone receptor-negative, and Her-2/neu negative). Its molecular genomic features are more similar to triple-negative breast cancer of no special type than to its salivary gland counterpart. However, the clinical course of the mammary acinic cell carcinoma appears to be less aggressive than the usual triple-negative breast carcinomas. [Ajkunic, A., et al. Breast (2022) 66:208-16].

The term “adenocarcinoma in situ” or AIS” as used herein refers to a condition in which abnormal cells are found in the glandular tissue, which may become a cancer and spread into nearby normal tissue.

The term “adjuvant therapy” as used herein refers to administration of additional therapy after primary surgery in order to kill or inhibit micrometastases.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer. In some embodiments, to A without B (optionally including elements other than B). In some embodiments, to B without A (optionally including elements other than A); in yet another embodiment, to both A and B (optionally including other elements); etc.

As used herein in the specification and in the claims, the term “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of”.

The term “artificial intelligence” or “AI” refers to the simulation of human intelligence by machines. AI programming includes the following cognitive skills: learning (acquiring data and creating rules for how to turn it into actionable information (algorithms), that provide step-by-step instructions for how to complete a specific task; reasoning (choosing the right algorithm); self-correction (fine-tuning algorithms to provide the best result); and creativity using AI techniques. Technologies that fall under the umbrella of AI include machine learning and deep learning.

The term “autoimmune disease” as used herein refers to a disease in which the pathology is caused by immune responses to self-antigens.

The term “Average Sale Price” or “ASP” as used herein refers to the average price charged by manufacturers to wholesalers net of any rebates or discounts.

The term “biomarker” (or “biosignature”) as used herein refers to peptides, proteins, nucleic acids, antibodies, genes, metabolites, or any other substances used as indicators of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (e.g., choices of drug treatment or administration regimes). In evaluating potential therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community. A “predictive biomarker” is a biomolecule that indicates therapeutic efficacy, i.e., an interaction that exists between the biomolecule and therapy that impacts patient outcome. A “prognostic biomarker” is a biomolecule that indicates patient survival independent of the treatment received. It is an indicator of innate tumor aggressiveness.

The term “biometrics” as used herein refers to measurable and unique biological (e.g., heartbeat, DNA, EEG), morphological (e.g., fingerprint mapping, palmprint mapping, hand geometry, car geometry, facial structure, retinal blood vessels, iris patterns, vein mapping), and behavioral characteristics or traits (e.g., voice recognition/voiceprint, speech patterns, breath pattern) used to authenticate identity.

The terms “cancer” or “malignancy” as used herein refer to diseases in which abnormal cells divide without control and can invade nearby tissues. Cancer cells also can spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in cells of the immune system. Central nervous system cancers are cancers that begin in the tissues of the brain and spinal cord.

The term “chemonaïve” as used herein means having not received prior chemotherapy.

The term “chemotherapy” as used herein refers to a treatment that uses drugs to stop the growth of cancer cells.

The term “clinical outcome” or “outcome” is used to refer to a specific result or effect that can be measured. Examples of outcomes include progression-free survival, overall survival, complete response, and stable disease.

The term “coherent” as used herein refers to a beam of photons that have the same frequency and do not spread and diffuse.

The terms “complete response” or “complete remission” or “CR” as used herein refer to the disappearance of all signs of cancer in response to treatment. This does not always mean the cancer has been cured.

The term “connecting” and its other grammatical forms as used herein refers to joining, being joined, or linking together.

The term “consolidation therapy”, also called “intensification therapy” and “post-remission therapy” as used herein refers to treatment that is given after cancer has disappeared following the initial therapy. Consolidation therapy is used to kill any cancer cells that may be left in the body.

The term “deep learning” as used herein, a subset of machine learning, is based on our understanding of how the brain is structured and involves use of artificial neural networks.

The term “Dependent Elements” as used herein refers to elements that have two behaviors. In the OMIC portion of RoseTra™, they act as Required Elements except that their appearance is dependent upon the values of the other Required Elements. For Breast Cancer, for example, both Oncotype DX Score and Lymphovascular Invasion are Dependent Elements. In the Treatment portion of RoseTra™, they behave as Switches in that their values can more precisely determine the Treatments that are presented. If applicable, unlike regular Switches, their values were entered in the OMIC portion and need not be acquired again in the Treatment section.

The term “DNA” or “deoxyribonucleic acid” as used herein refers to a polynucleotide formed from covalently linked deoxyribonucleotide units. DNA serves as the store of hereditary information within a cell and the carrier of this information from generation to generation.

The term “dose intensity” as used herein is defined as the amount of drug delivered per unit of time, expressed as mg/m2/week, regardless of the schedule or route of administration. This is simply a method of comparing a total dose given over a period of time between or among treatment protocols. The term “delivered dose intensity” or “DDI” as used herein is delivered total dose (in mg/m2)/standard time to complete chemotherapy (in days).

The term “ECOG performance status scale” as used herein refers to a scale used to assess how a patient's disease is progressing, assess how the disease affects the daily living abilities of the patient, and determine appropriate treatment and prognosis. The scale was developed by the Eastern Cooperative Oncology Group (ECOG), now part of the ECOG-ACRIN Cancer Research Group, Oken M, Creech R, Tormey D., et al. Toxicity and response criteria of the Eastern Cooperative Oncology Group. Am J Clin Oncol. (1982) 5:649-655:

TABLE 1
ECOG performance status scale

| Grade | ECOG Performance Status |
|---|---|
| 0 | Fully active, able to carry on all pre-disease performance |
| | without restriction |
| 1 | Restricted in physically strenuous activity but ambulatory |
| | and able to carry out work of a light or sedentary nature, |
| | e.g., light housework, office work |
| 2 | Ambulatory and capable of all self-care but unable to carry |
| | out any work activities; up and about more than 50% of |
| | waking hours |
| 3 | Capable of only limited self-care; confined to bed or |
| | chair more than 50% of waking hours |
| 4 | Completely disabled; cannot carry on any self-care; |
| | totally confined to bed or chair |
| 5 | Dead |

The term “electromagnetic spectrum” as used herein refers to the range of wavelengths or frequencies over which electromagnetic radiation extends. Electromagnetic energy travels in waves and is divided into seven broad ranges by frequency. The ranges, from lowest to highest frequency, are radio waves, microwaves, infrared, visible, ultraviolet, x-rays and gamma rays. The human eye can only detect a small portion of this spectrum, called visible light, corresponding to wavelengths from 380 nm to 700 nm. The size of an electromagnetic wave is determined by its wavelength or its frequency.

The term “emission spectrum” as used herein refers to a pattern of frequencies (or wavelengths) emitted by a source. The term “line emission spectrum” as used herein refers to the pattern of lines seen when light from a substance is separated out into its different wavelengths.

The term “EndoPredict Test” as used herein refers to a genomic test for human subjects newly diagnosed with early-stage, estrogen-receptor-positive, HER2-negative breast cancer.

The term “engine” as used herein refers to the part of a computer program that implements a technique.

The term “epigenetics” as used herein refers to heritable traits that are not a consequence of changes in gene sequence. These traits are the result of alterations in gene expression regulated by changes in DNA accessibility or chromatin structure. Epigenetic modifications, or tags, that lead to changes in DNA accessibility can be brought about by DNA methylation, posttranslational modification of histone proteins, or noncoding RNA actions in the nucleus. [Loscalzo, J. and Handy, D E. Pulmonary Circulation (2014) 4 (20:169-74, citing Handy, D E., et al. Circulation (2011) 123:2145-56).

The term “exon” as used herein refers to a segment of a eukaryotic gene that consists of a sequence of nucleotides that will be represented in messenger RNA or the final transfer RNA or ribosomal RNA. In protein-coding genes, exons encode amino acids in the protein. An exon is usually adjacent to a noncoding DNA segment called an intron.

As used herein, the term “expression” is meant to encompass production of an observable phenotype by a gene, usually by directing the synthesis of a protein. It includes the biosynthesis of mRNA, polypeptide biosynthesis, polypeptide activation, e.g., by post-translational modification, or an activation of expression by changing the subcellular location or by recruitment to chromatin.

The term “frequency of a wave” as used herein refers to the number of oscillations, or movements back and forth, per unit time.

The term “gene” as used herein refers to a region of DNA that controls a discrete hereditary characteristic, usually corresponding to a single protein or RNA. This definition includes the entire functional unit, encompassing coding sequences, noncoding regulatory sequences and introns.

The term “human epidermal growth factor receptor-2” (HER2, also called HER2/neu, c-erB-2, a human EGF receptor 2) as used herein refers to a membrane tyrosine kinase and oncogene that is overexpressed in some types of cancer cells, including breast, ovarian, bladder, pancreatic, stomach, and esophageal cancers. When activated, it provides the cell with potent proliferative and anti-apoptosis signals, and it is a major driver of tumor development and progression.

The term “histology” as used herein refers to a field of biology and medicine dedicated to elucidating tissue structure, function, and disease states. The principal techniques of histology involve the use of various chemical stains to interrogate tissue samples. The term “histopathology” as used herein involves the clinical application of histological methods to examine diseased cells and tissues for diagnostic or prognostic analysis of various medical conditions.

The International Statistical Classification of Diseases and Related Health Problems 10th Revision (ICD-10) is a coding of diseases, signs and symptoms, abnormal findings, complaints, social circumstances and external causes of injury or diseases as classified by the World Health Organization (WHO). These code sets, which are considered classification code sets, are at a higher level of information than some other medical code sets like the Systematized Nomenclature of Medicine (SNOMED), which is used by federal government systems for the electronic exchange of clinical health information.

The term “immune checkpoint molecules” as used herein refers to ligand-receptor pairs that exert inhibitory or stimulatory effects on immune responses. Examples include programmed cell death 1 receptor (PD-1, also known as CD279), thought to regulate T cell proliferation later in the immune response, and its ligand programmed cell death ligand 1 (PD-L1), lymphocyte-activation gene 3 (LAG3), which suppresses T cells activation and cytokine secretion, thereby ensuring immune homeostasis and shows synergy with PD-1 to inhibit immune responses (Long, L., et al. Genes Cancer (2018) 9 (5-6): 176-89) and cytotoxic T-lymphocyte-associated antigen 4 (CTLA4; also known as CD152), a negative regulator of T cell immune function thought to regulate T cell proliferation early in an immune response [Buchbinder, E I, and Desai, A. Am. J. Clin. Oncol. (39) (10): 98-106). In addition, glucocorticoid-induced TNFR family related gene (GITR), a member of the TNFR superfamily (TNFRSF) that is expressed in different cell types, including T lymphocytes activation; GITR activation by its ligand (GITRL) influences the activity of effector and regulatory T cells, thus participating in the development of immune response against tumors and infectious agents, as well as in autoimmune and inflammatory diseases. [Nocentini, G., et al. Br. J. Pharmacol. (2012) 165 (7): 2089-99]. T-cell immunoglobulin and mucin domain 3 (Tim-3) is a checkpoint receptor expressed by a wide variety of immune cells as well as leukemic stem cells. [Acharya, N., et al. J. Immunother. Cancer (2020) 8(10: e000911). T-cell immunoreceptor with immunoglobulin and ITIM domains (TIGIT) is an immune checkpoint receptor that can suppress T-cell activation and promote T-cell exhaustion. Inhibition of TIGIT may increase cytotoxic T-cell proliferation and function. Inducible T cell costimulator (ICOS, cluster of differentiation (CD278)) is an activating costimulatory immune checkpoint expressed on activated T cells. Its ligand, ICOSL is expressed on antigen-presenting cells and somatic cells, including tumor cells in the tumor microenvironment. [Solinas, C., et al. ESMO Open. (2020) 5(1): e000544].

The term “immunotherapy” as used herein refers to measures taken using immunological methods and principles to target the hyper- or hypo-immune state of an organism, intervene or adjust the organism's immune function artificially, and strengthen or attenuate the immune response so as to treat disease. For example, cancer immunotherapy can enhance the immune system's ability to recognize, target and eliminate cancer cells in the body. [Zhang, Z., et al. Front. Immunol. (2021) 12: Barbari, C., et al. Intl J. Mol. Sci. (2020) 21: 5009]. Some types of immunotherapy only target certain cells of the immune system. Others affect the immune system in a general way.

The term “induction therapy”, also called first line therapy, primary therapy and primary treatment, refers to the first treatment given for a disease.

The term “intensity” as used herein with regard to light or number of photons per second is the ratio of power to unit area. It is a measure of how much energy is emitted at a given moment and can also be thought of as a measure of brightness. The intensity (power per unit area) of a monochromatic light beam in quantum physics is the number of photons per unit area per unit time, multiplied by the energy per photon.

The term “interface” as used herein refers to a connection between two systems through which information is exchanged.

The term “invasive” as used herein refers to a cancer that has spread beyond the layer of tissue in which it developed and is growing into surrounding, healthy tissues.

The term “invasive cribriform carcinoma” as used herein refers to a breast cancer in which the cancer cells invade the stroma (connective tissues of the breast) in nestlike formations between the ducts and lobules. Within the tumor, there are distinctive holes in between the cancer cells.

The term “Ki67” or antigen Ki-67, also known as Ki-67 or Marker of Proliferation Ki-67 (MKI67), refers to a protein in humans encoded by the MKI67 gene [Davey, M G et al. Cancers (Basel) 13 (17): 4455, citing Schonk, D M., et al. Hum. Genet. (1989) 83: 297-99]. Ki-67 encodes two protein isoforms with molecular weights of 345 and 395 kilodaltons and was initially identified in Hodgkin lymphoma cell nuclei [Id., citing Scholzen, T. and Gerdes, J. J. Cell Physiol. (2000) 182:311-322]. Ki-67 remains active during the G1, S, G2, and M phases of the cell cycle [33], making it a marker of cell proliferation [Id., citing Shirendeb U., et al. Acta Histochem. Cytochem. (2009) 42:181-90; Hooghe, B, et al. Nucleic Acids Res. (2008) 36: W128-W132] and an accepted hallmark of oncogenesis [Id., citing Gutschner, T. and Diederichs, S. RNA Biol. (2012) 9:703-19]. During interphase, the Ki-67 antigen can be exclusively detected within cell nuclei, whereas in mitosis, most of the protein is relocated to the surface of cellular chromosomes [Id., citing Cuylen, S., et al. Nature (2016) 535: 308-12]. Ki-67 remains absent during the quiescent G0 phase, and levels reduce significantly during anaphase and telophase [Id., citing Modlin, I M., et al. J. Natl Cancer Inst. (2008) 100: 1282-9]. Immunohistochemical evaluation of Ki-67 is now incorporated into the paradigm for several cancer types due to its reliable correlation with the proliferative activity of cancer cells [Id., citing Miller, I., et al. Cell Rep. (2018) 24: 1105-12]. Reliable prognostication using Ki-67 as a solitary biomarker has been validated in a number of cancers, including breast, prostate, cervical, lung, soft tissue, neuroendocrine cancers, and gastrointestinal stromal tumors [Id., citing Ishihara, M., et al. Oncology (2013) 84: 135-40; Sorbye, S W., et al. PLOS ONE (2012) 7: e47068; Ciancio, N., et al. Multidiscip. Respir. Med. (2012) 7: 29; Josefsson, A., et al. Scand. J. Urol. Nephrol. (2012) 46: 247-57; Zhao, W-Y, et al. Int. J. Clin. Exp. Pathol. (2014) 7:2298-2304; Nadler, A., et al. Virchows Arch. (2013) 462: 501-5]. Nevertheless, this biomarker has not been completely integrated as a standard component of clinical decision making or pathological reporting (Id., citing Denkert, C., et al. Breast (2015) 24 (Suppl. 2): S67-S72], largely due to inconsistencies in its scoring.

The term “lymphovascular invasion” or “LVI” as used herein refers to the presence or absence of tumor cells in lymphatic channels (not lymph nodes) or blood vessels within the primary tumor as noted microscopically by a pathologist. Traditionally, lymphovascular invasion (LVI) has represented one of the foremost pathological features of malignancy and has been associated with a worse prognosis in different cancers, including breast carcinoma.

The term “laser” as used herein stands for Light Amplification by Stimulated Emission of Radiation. Lasers are basically excited light waves. Laser light is monochromatic, directional, and coherent (meaning the light waves are identical and in phase, which produces a beam of coherent light. There are many types of lasers that use gases, such as helium, neon, argon, and carbon dioxide. Exemplary lasers also use semiconductors (e.g., galiodium and arsenic), solid-state material (e.g., ruby, glass), and chemicals (e.g., hydrofluoric acid) in their operation.

The term “machine learning” as used herein refers to a subset of artificial intelligence and computer science that uses algorithms that learn from data to make predictions. These predictions can be generated through supervised learning, where algorithms learn patterns from existing data, or unsupervised learning, where they discover general patterns in data.

The term “MammaPrint®” (or “MP”), made by Agendia, is a 70-gene signature that assesses the 10-year risk of distant metastasis in breast cancer tissue of early-stage (stage I, II or IIIA) invasive breast cancer patients that is less than 5 cm and is either node-negative or has spread to 1 to 3 lymph nodes. This assay classifies tumors into low- and high-risk of distant relapse and has been shown to identify patients who may safely avoid chemotherapy.

The term “metastasis” as used herein refers to spread of cancer cells from the place where they first formed to another part of the body. In metastasis, cancer cells break away from the original (primary) tumor, travel through the blood or lymph system, and form a new tumor in other organs or tissues of the body. The new, metastatic tumor is the same type of cancer as the primary tumor. For example, if breast cancer spreads to the lung, the cancer cells in the lung are breast cancer cells, not lung cancer cells.

The term “microRNA,” “miRNA”, or “miR” as used herein refers to a class of small, non-coding RNA molecules, usually from about 18 to about 28 nucleotides in length. MicroRNAs are partially complementary to one or more messenger RNA (mRNA) molecules, and function in posttranscriptional regulation of gene expression and RNA silencing.

The term “minimum residual disease” or “measurable residual disease” or “MRD” are used interchangeably to refer to the small number of cancer cells that remain in a patient that share phenotypic similarity (e.g., histologic appearance, lineage markers) and genetic heritage (e.g., mutations and rearrangements) with the original tumor cells. This definition excludes residual cells that harbor somatic alterations and/or phenotypic alterations but are not fully malignant. [Luskin, M R., et al. Nat. Rev. Cancer (2018) 18 (4): 255-63].

The term “modality” as used within healthcare refers to a method of treatment, a piece of equipment, or an interventional strategy. Examples of modalities in oncology include, without limitation, chemotherapy, radiation therapy or surgery; devices and equipment utilized within medical imaging, e.g., X-rays, magnetic resonance imaging (MRI), ultrasound; CT scans.

The term “monochromatic” as used herein refers to light emitted at a single pure frequency or wavelength.

The term “mutation” as used herein refers to a change of the DNA sequence within a gene or chromosome of an organism resulting in the creation of a new character or trait not found in the parental type, or the process by which such a change occurs in a chromosome, either through an alteration in the nucleotide sequence of the DNA coding for a gene or through a change in the physical arrangement of a chromosome. Three mechanisms of mutation include substitution (exchange of one base pair for another), addition (the insertion of one or more bases into a sequence), and deletion (loss of one or more base pairs).

The term National Comprehensive Cancer Network® (NCCN®) as used herein refers to a not-for-profit alliance of 33 leading cancer centers in the United States devoted to patient care, research, and education. NCCN Guidelines are used by clinicians around the world as a standard resource for clinical decision-making. The NCCN Harmonized Guidelines™ are targeted regional resources created as part of a collaborative effort to combat the skyrocketing cancer rates and unique circumstances of cancer care.

The term “neoadjuvant therapy” as used herein refers to a type of induction therapy delivered before primary treatment to help increase chance of success and decrease risk of recurrence.

The term “non-coding RNAs (ncRNAs”) as used herein refers to functional RNA molecules that are transcribed from DNA but not translated into proteins. High-throughput sequencing technology confirmed that over 98% of the human genome is transcribed into ncRNAs, which are divided into two main groups: the small non-coding RNAs (<200 nucleotides) and the long non-coding RNAs (lncRNAs) (>200 nucleotides). In general, ncRNAs play a role in hetero-chromatin formation, histone modification and DNA methylation, leading to regulating gene expression at the transcriptional and post-transcriptional level. Epigenetic related ncRNAs include miRNA, siRNA, piRNA and lncRNA. Non-coding RNA types are summarized in Table 2.

TABLE 2

| RNA | Functions | Coding | Typical Size | |
|---|---|---|---|---|
| microRNA | Post-transcriptional | No | 17-24 | nt |
| (miRNA) | gene silencing | | | |
| Y RNA | Component of Ro60 | No | ≈100 | nt |
| | ribonucleoprotein | | | |
| | particle; initiation | | | |
| | factor for DNA | | | |
| | replication | | | |
| Signal Recognition | Component of SRP | No | ≈280 | nt |
| particle RNA (SRP | ribonucleoprotein | | | |
| RNA) | complex that directs | | | |
| | protein trafficking | | | |
| Transfer RNA | Adapter for matching | No | 76-90 | nt |
| (tRNA) | amino acid to mRNA | | | |
| Ribosomal RNA | RNA component of | No | 185 (1.9 kb) 28 S | |
| (rRNA) | ribosomes | | (5.0 kb) | |
| Small nuclear RNA | RNA processing such | No | ≈150 | nt |
| (snRNA) | as mRNA splicing | | | |
| Small nucleolar | Guiding chemical | No | 20-24 | nt |
| RNA (snoRNA) | modifications of | | | |
| | other RNAs | | | |
| Long noncoding | Many, including in- | No | >100 | nt |

| | |
|---|---|
| RNA (lncRNA) | transcription and |
| | post-transcription |
| | regulation |

The term “objective response rate” or “ORR” as used herein refers to the percentage of patients in a study or treatment group who have a partial response or complete response to the treatment within a certain period of time. It does not include stable disease.

The term “oncogene” as used herein refers to a mutated version of a proto-oncogene. Oncogenes, however, typically exhibit increased production of these proteins, thus leading to increased cell division, decreased cell differentiation, and inhibition of cell death; taken together, these phenotypes define cancer cells. Oncogenes arise as a result of mutations that increase the expression level or activity of a proto-oncogene. Underlying genetic mechanisms associated with oncogene activation include, without limitation, point mutations, deletions, or insertions that lead to a hyperactive gene product; point mutations, deletions, or insertions in the promoter region of a proto-oncogene that lead to increased transcription; gene amplification events leading to extra chromosomal copies of a proto-oncogene; chromosomal translocation events that relocate a proto-oncogene to a new chromosomal site that leads to higher expression; and/or chromosomal translocations that lead to a fusion between a proto-oncogene and a second gene, which produces a fusion protein with oncogenic activity. Examples of oncogenes and their associated cancers are shown in Table 3.

TABLE 3
Examples of oncogenes associated with certain cancers

| Oncogene | Disease |
|---|---|
| HER-2 | Breast cancer |
| BCR/ABL | Chronic myeloid leukemias |
| C-KIT | Gastrointestinal stromal tumor |
| EGFR | NSCLC, Head and neck; colorectal, Pancreas |
| VEGF | Breast, colorectum; kidney |
| VEGFR, B-RAF | Kidney |

The term “Oncotype DX Breast Recurrence Score® test” as used herein refers to a multigene assay developed for patients with early-stage hormone receptor positive (HR+), HER2 negative breast cancer The Oncotype DX Breast Recurrence Score® report provides three pieces of information to guide adjuvant chemotherapy treatment decisions for node-negative and node-positive, hormone receptor-positive, HER2-negative, early-stage breast cancer patients. First, the Recurrence Score® result, which is a number between 0 and 100, is calculated by measuring the activity of specific genes in the breast cancer tissue. It is used to predict the risk of the breast cancer returning at a distant site, distant recurrence and whether chemotherapy may help reduce the risk. Second, a distant recurrence risk at 9 years (percentage) shows the individualized risk of distant recurrence within 9 years based on the Recurrence Score result when treated with hormonal therapy alone for 5 years. Third, a Group average absolute chemotherapy benefit (percentage) indicates the benefit expected from adding chemotherapy to hormonal therapy in order to reduce the risk of breast cancer recurrence or death for the Recurrence Score risk group.

The term “overall survival” as used herein according to RECIST refers to the length of time from either the date of diagnosis or the start of treatment for a disease, such as cancer, to time of death from any cause.

The term “PALB2” as used herein refers to a gene in the homologous recombination repair (HRR) pathway of the DNA damage response (DDR)

The term “partial response” as used herein according to RECIST refers to at least a 30% decrease in the sum of the target lesions.

The terms “Pathologic Complete Response” or “Pathologic Complete Remission” are used interchangeably herein to refer to the lack of all signs of cancer in tissue samples removed during surgery or biopsy after treatment with radiation or chemotherapy. To find out if there is a pathologic complete response, a pathologist checks the tissue samples under a microscope to see if there are still cancer cells left after the anticancer treatment. Knowing if the cancer is in pathologic complete response may help show how well treatment is working or if the cancer will recur.

The term “phenotype” as used herein refers to qualitative and quantitative observable characteristics of cells. A cell's phenotype is the culmination of several cellular processes through a complex network of molecular interactions that ultimately result in a unique morphological signature. Clinical, biochemical and imaging methodologies can be used to refine and characterize a phenotype.

The term “photon” as used herein refers to a minute discrete bundle of electromagnetic energy. For example, a photon is produced when an electron excited to a higher-than-normal orbit in a chemical atom falls back to its regular orbit. A photon has no rest mass, travels at the speed of light in a vacuum, and is electrically neutral, i.e., it is not deflected by electric and magnetic fields. In a laser beam, photon beams move in the same direction at the same wavelength; this is achieved by transmitting the energized electrons through an optical “gain medium”, such as glass or a gas.

The term “plug-in” as used herein refers to an accessory program that provides additional functions for a main application program.

The term “point of care” (or POC″) as used herein refers to the location where healthcare services are provided. This can include a variety of settings, such as a doctor's office, clinic, hospital, or a patient's home. The term “point of care” is used to emphasize that healthcare services should be delivered in the most appropriate and convenient location for the patient.

The term “point-of-care technology” refers to the devices, information systems, and other software solutions that are used to deliver healthcare services at the point of care. This can include electronic health records (EHRs), point-of-care connection platforms, diagnostic devices, and other health applications. The goal of point-of-care technology is to improve the efficiency, quality, and safety of healthcare delivery.

The term “post-translational modifications” (or “PTMs”) refer to mechanisms used by cells to diversify and extend their protein functions beyond what is dictated by protein-coding sequences in the genome. These chemical reactions range from the addition of small moieties, such as phosphate (phosphorylation), complex biomolecules, as in glycosylation, to proteolytic cleavage [Landini, A., et al. Nature Communications (2022) 13: Article 1586, citing Deribe, Y L et al. Nat. Struct. Mol. Biol. (2010) 17:666-72]. PTMs alter the structure and properties of proteins and are thus involved in the dynamic regulation of most cellular events. N-glycosylation is one of the most common protein PTMs, where carbohydrate structures called glycans are covalently attached to an asparagine (Asn) residue of a polypeptide backbone. The PTMs that have taken place in histone proteins can affect gene expression by altering chromatin structure. Histone modifications act in varied biological processes such as transcriptional activation/inactivation, chromosome packaging, mitosis, meiosis, apoptosis, and DNA damage/repair. [Ramazi, S., et al. J. Biosci. (2020) 45: 135].

The term “prior authorization” (sometimes called preauthorization or precertification) as used herein refers to a health plan cost-control process by which physicians and other health care providers must obtain advance approval from a health plan before a specific service is delivered to the patient to qualify for payment coverage.

The term “prognosis” as used herein refers to a prediction of the likely outcome or course of a disease and the chance of recovery or recurrence.

The term “progression” as used herein refers to the course of disease as it becomes worse or spreads in the body.

The term “progressive disease” as used herein according to RECIST refers to at least a 20% increase in the sum of diameters of up to 5 target lesions (2 lesions/organ), taking as reference the smallest sum on study and an absolute lesion increase of at least 5 mm or the appearance of new lesions.

The term “progression-free survival” as used herein according to RECIST refers to the time from randomization or beginning of treatment until objective tumor progression or death.

The “Prosigna Breast Cancer Prognostic Gene Signature Assay” (formerly called the PAM50 test), made by Veracyte, is a genomic test that analyzes the activity of certain genes in early-stage, hormone-receptor-positive breast cancer. It can only be used on breast cancers diagnosed in postmenopausal women that: are stage I or stage II and lymph node-negative; are stage II with one to three positive nodes; are hormone-receptor-positive; are invasive; and have been treated with surgery and hormonal therapy. The Prosigna assay is performed on preserved tissue that was removed during the original biopsy or surgery.

The term “proto-oncogene” as used herein refers to a group of genes that cause normal cells to become cancerous when they are mutated. Often, proto-oncogenes encode proteins that function to stimulate cell division, inhibit cell differentiation, and halt cell death. All of these processes are important for normal human development and for the maintenance of tissues and organs.

As used herein, the term “real-time” or “real time” means without perceivable delay or information that is delivered immediately after collection or processing. These terms also include a time delay introduced by automated processing (e.g., near real-time).

The term “Real World Evidence” or “RWE” as used herein refers to evidence from real world data sources such as electronic health records (EHR).

The term “receptor” as used herein refers to specialized proteins that bind a specific extracellular signal molecule (ligand) and initiates a response in the cell. Cell surface receptors are located in the plasma membrane, with their ligand-binding site exposed to the external medium. Intracellular receptors bind ligands that diffuse into the cell across the plasma membrane.

The term “RECIST” or “Response Evaluation Criteria In Solid Tumors” as used herein refers to a standard way to measure how well a cancer patient responds to treatment. It is based on whether tumors shrink, stay the same, or get bigger. To use RECIST, there must be at least one tumor that can be measured on x-rays, CT scans, or MRI scans. The types of response a patient can have are a complete response (CR), a partial response (PR), progressive disease (PD), and stable disease (SD). [Eisenhauer, E A., et al. Eur. J. Cancer (2009) 45 (2): 228-47]. Major limitations of RECIST that universally affect the response assessment regardless of tumor types or agents include variability of tumor size measurements and tumoral heterogeneity both within a lesion and among different lesions in a patient. [Nishino, M. AM. Socy Clinical Oncol. Edu. Book. (2018) 38: 1019-29]. The term “modified RECIST (mRECIST) criteria” only concerns hepatocellular carcinoma and only takes into account the viable portion defined as the contrast-enhanced portion of the tumor on hepatic arterial phase images. [Yu, H., et al. BMJ Open (2022) 12 (6): e052294].

The term “recurrent cancer” or “recurrence” means a cancer that has come back, usually after a period of time during which the cancer could not be detected. The cancer may come back to the same place as the primary tumor or to another place in the body.

The term “refractory cancer” or “resistant cancer” means a cancer that does not respond to treatment. The cancer may be resistant at the beginning of treatment, or it may become resistant during treatment.

The term “relapse” refers to the return of a disease or the signs and symptoms of a disease after a period of improvement.

The terms “relapse-free survival” (RFS) or “disease-free survival” (DFS) mean the length of time after primary treatment for a cancer ends that the patient survives without any signs or symptoms of that cancer.

The term “Required Elements” as used herein are the set of Patient Specific, Disease Specific (PSDS) characteristics whose values are necessary in order to generate an OMIC.

The term “RNA” or ribonucleic acid” as used herein refers to a polymer formed from covalently linked ribonucleotide monomers. The term “messenger RNA”, for example, refers to a single stranded RNA molecule in cells that carries genetic information from DNA in the nucleus to the cytoplasm where it specifies the amino acid sequence of a protein. It is produced by RNA splicing in eukaryotes from a larger RNA molecule made by RNA polymerase as a complementary copy of DNA. It is translated into protein in a process catalyzed by ribosomes, which are particles composed of ribosomal RNAs and ribosomal proteins that associate with messenger RNA and catalyze the synthesis of protein.

The term “solid tumor” as used herein refers to an abnormal mass of tissue that usually does not contain cysts or liquid areas. Solid tumors may be benign (a growth that does not invade nearby tissue or spread to other parts of the body) or malignant (meaning to grow in an uncontrolled way); malignant tumors can invade nearby tissues and spread to other parts of the body through the blood and lymph system). Different types of solid tumors are named for the type of cells that form them. Types of solid tumors are sarcomas, carcinomas, and lymphomas; leukemias (cancers of the blood) generally do not form solid tumors.

For example, a carcinoma is a cancer that begins in the skin or in tissues that line or cover internal organs. An adenocarcinoma is a cancer that forms in the glandular tissue that lines certain internal organs and makes and releases substances in the body, such as mucus, digestive juices, and other fluids. Most cancers of the breast, lung, esophagus, stomach, colon, rectum, pancreas, prostate, and uterus are adenocarcinomas.

A lymphoma is a malignant solid neoplasm of the lymphoid system, which produces immune cells. Abnormal lymphocytes become lymphoma cells, which multiply and collect in the lymph nodes. Over time, these cancerous cells impair the immune system. There are two categories of lymphomas: Hodgkin lymphoma and non-Hodgkin lymphoma. About 12 percent of people with lymphoma have Hodgkin lymphoma. Most non-Hodgkin lymphomas are B-cell lymphomas, and either grow quickly (high-grade) or slowly (low-grade). There are over a dozen types of B-cell non-Hodgkin lymphomas. The rest are T-cell lymphomas.

A sarcoma is a type of cancer that begins in bone or in the soft tissues of the body, including cartilage, fat, muscle, blood vessels, fibrous tissue, or other connective or supportive tissue. Bone and soft tissue sarcomas are the main types of sarcoma. Soft tissue sarcomas can develop in soft tissues like fat, muscle, nerves, fibrous tissues, blood vessels, or deep skin tissues. They can be found in any part of the body. Most of them start in the arms or legs. They can also be found in the trunk, head and neck area, internal organs, and the retroperitoneum. The different types of sarcoma are based on where the cancer forms. For example, osteosarcoma forms in bone, liposarcoma forms in fat, and rhabdomyosarcoma forms in muscle.

The term “social determinants of health” or “SDOH” as used herein refers to nonmedical factors that influence health outcomes. According to the Center for Disease Control (CDC), the United States' science-based, data-driven, service organization that protects the U.S. public's health, they are the conditions in which people are born, grow, work, live and age. Five key areas of SDOH are: healthcare access and quality; education access and quality; social and community context; economic stability; and neighborhood and built environment. (https://www.cdc.gov/about/priorities/why-is-addressing-sdoh-important.html, visited May 29, 2024).

The term “somatic genotype” as used herein refers to the genetic constitution of an individual cell or organism that is not a germ cell (meaning a precursor cell that will give rise to gametes, e.g., eggs or sperm).

The term “somatic mutation” as used herein refers to a genetic alteration that occurs after conception to any of the cells of the body and that is not a germ cell (e.g., egg or sperm cell).

The term “stable disease” as used herein refers to fitting the criteria neither for progressive disease nor for a partial response.

The term “translocation” as used herein refers to a type of chromosomal abnormality in which a chromosome breaks and a portion of it reattaches to a different chromosomal location.

The term “tumor grade” as used herein and described in Table XX refers to how normal or abnormal cancer cells look under a microscope. The more normal the cells look, the less aggressive the cancer and the more slowly it grows and spreads. The more abnormal the cells look, the more aggressive the cancer and the faster it is likely to grow and spread. Although systems for describing tumor grade can differ depending on the type of cancer, most tumors are graded as X, 1, 2, 3 or 4 as shown in Table 4.

TABLE 4
Histologic Grade (G)

| | |
|---|---|
| GX | Grade cannot be accessed |
| G1 | Well differentiated |
| G2 | Moderately differentiated |
| G3 | Poorly differentiated |
| G4 | Undifferentiated |

The term “tumor marker” as used herein refers to measurable biochemical that are either produced by tumor cells (tumor-derived) or by the body in response to tumor cells (tumor-associated). They are typically substances that are released into the circulation and thus measured in the blood. Genomic markers (such as tumor gene mutations, patterns of tumor gene expression, and nongenetic changes in tumor DNA) are being used as tumor markers. Tumor markers can help to diagnose cancer, identify the type of cancer; the stage of the cancer; estimate prognosis; identify biomarkers for cancer treatment; indicate whether the tumor is responding to treatment; check for recurrence.

The term “tumor stage” as used herein refers to the extent of a cancer, meaning how large a tumor is and how far the cancer has spread. A cancer is always referred to by the stage it was given at diagnosis, even if it changes over time. New information about how a cancer has changed over time is added to the original stage. Stages I, II and III indicate cancer is present. The higher the number, the more advanced the cancer is. Stage IV indicates that the cancer has spread to distant parts of the body.

Tumor staging requires the collaborative effort of many professionals, including the managing physician, pathologist, radiologist, and others. While the pathologist, radiologist, and other health care providers generate important staging information and may contribute important T-related, N-related, and/or M-related information, tumor stage is ultimately defined from the synthesis of an array of patient history and physical examination findings supplemented by imaging and pathology data. Only the treating physician (or the managing physician[s] with complete access to the full set of patient information) can assign the patient's stage, because only (s)he routinely has access to all of the pertinent information from physical examinations, imaging studies, biopsies, diagnostic procedures, surgical findings, and pathology reports.

The terms “staging system”, “AJCC staging system”, or TNM staging system” are used interchangeably herein to refer to a classification system for evaluating cancer at a population level in terms of the anatomic extent of disease. The AJCC staging system was created and is updated by the American Joint Committee on Cancer (AJCC) and the International Union Against Cancer (UICC). The AJCC staging system is used to describe most types of cancer. It describes the amount and spread of cancer in a patient's body, using T to describe, the size of the tumor and any spread of cancer into nearby tissue; N to describe spread of cancer to nearby lymph nodes; and M to describe metastasis (spread of cancer to other parts of the body).

The term “variant” is used herein to refer to nucleotide or polypeptide sequences with substantial identity to a reference nucleotide or polypeptide sequence. The differences in the sequences may be the result of changes in sequence or structure.

The term “wavelength” as used herein refers to the distance between two successive crests of a wave.

### Embodiments

Embodiments of the present disclosure provide methods, systems and computer readable media that, in real time and at the point of care, focus healthcare information that characterizes a patient and the patient's disease state down to what is relevant to guide treatment of that patient, which results in the optimal clinical outcome, and then provides an evidence-based cost-optimized path of care.

Some embodiments include methods and systems that receive user input regarding individual patient data/medical records. Some embodiments include methods and systems that focus healthcare information that characterizes a patient and the patient's disease state down to what is relevant to guide treatment of that patient that results in an optimal clinical outcome.

Some embodiments provide an evidence-based cost-optimized path of care by deriving a breakdown of a one-year cost of care comprising every element of care of each treatment regimen over a one-year period for that patient.

Outputs of the methods and systems include, without limitation, indicating to a healthcare provider (e.g., healthcare practitioner, hospital system, insurance company) a cost-optimized path of care; providing a basis for price negotiation of drugs to optimize average sale price while maximizing rebates; lowering total cost of care and generating shared savings; and enabling monitoring and reporting of the relevant economic outcomes and relevant clinical outcomes.

### Method

According to one aspect, the present disclosure provides a method (100) for identifying and presenting at a point of care and in real time a care-optimized evidence-based path of care for a patient with a disease comprising:
(1) determining treatment choices based on evidence-based paths of care deemed appropriate for the patient by:
(a) accessing a plug-in comprising an interface that accepts a patient specific disease-specific characterization code (PSDSCC);
(b) translating by communication with an action code engine the patient disease-specific characterization code into an action code, wherein the action code links to actions (meaning how a patient is treated) by specifying a set of associated switches;
(c) generating, from values of the action code and the set of associated switches, a set of clinically equivalent candidate generic treatment regimens comprising interchangeable regimens for patients that exhibit the disease specific characterization elements presented, wherein drugs are listed by class, i.e., no specific drugs are identified at this stage;
(2) optimizing by communication with a care-optimization engine, costs of all aspects of care that are changeable in the candidate generic treatment regimen to care for a specific disease by
(a) identifying, characterizing, cost-assessing and either avoiding or reducing its cost; and
(b) returning an actual treatment regimen comprising a most cost-effective treatment plan comprising specific drugs substituted for the generic drug classes.
(3) presenting by display on a display device at the point of care at a time of treatment decision:
(i), the evidence-based actual treatment regimen comprising the evidenced path of care associated with a best expected clinical outcome and a ranked total cost of care; and
(ii) an ideal value-based path of care;
(4) collecting, analyzing, and presenting by display economic outcome data and relevant clinical outcome data for monitoring and reporting the data over time; and
(5) optimizing drug utilization in accordance with site-specific purchasing contracts that include acquisition cost rebates and other economic benefits (for example, lower acquisition cost and lower administrative cost through removal of precertification and prior authorization) to enhance practice economic margins and reduce payor cost through reducing total cost of care and delivering the expected relevant clinical outcome.

In some embodiments, the disease is any clinical condition, e.g., a cardiovascular disease, a metabolic disease (e.g., diabetes), an immune mediated disease (e.g., an autoimmune disease, rheumatology), organ transplantation; neurologic and neurodegenerative disorder; renal and bladder disorders, a musculoskeletal disorder, a pulmonary disease, a hematologic disorder, a cutaneous disorder, an infectious disease, digestive and hepatic disorders, behavioral health disorders, and disorders of childbirth.

For example, in some embodiments where the disease is in ophthalmology, the intervention is a change from a more to a less expensive treatment plan, and the clinical outcome is preservation of vision. In some embodiments, where the disease is in rheumatology, the intervention is delaying or avoiding switching from methotrexate to a biologic, and the outcome is maintaining or improving joint severity score.

In some embodiments, the disease is a cancer. In some embodiments, the cancer comprises a solid tumor. Examples of cancers by body location/system include AIDS-related cancers; breast, digestive/gastrointestinal; endocrine and neuroendocrine; eye; genitourinary; germ cell; gynecologic; head and neck; hematologic/blood; musculoskeletal; neurologic; respiratory/thoracic; skin; and unknown primary. Common cancer types include, without limitation, bladder cancer, breast cancer, colon and rectal cancer; endometrial cancer; kidney cancer; leukemia; liver cancer; lung cancer; melanoma; non-Hodgkin lymphoma; pancreatic cancer; prostate cancer; and thyroid cancer.

In the exemplified embodiment, the patient is a patient with breast cancer. Breast cancer is now recognized as a heterogeneous disease with varied morphology, molecular features, tumor behavior, and response to therapeutic strategies. These parameters are underpinned by a combination of genomic and immunohistochemical tumor factors, with estrogen receptor (ER) status, progesterone receptor (PgR) status, human epidermal growth factor receptor-2 (HER2) status, Ki-67 proliferation indices, and multigene panels all playing a contributive role in the substratification, prognostication and personalization of treatment modalities for each case. [Davey, M G., et al. Cancers (Basel) (2021) 13 (17): 4455].

Breast cancer accounts for 12.5% of all new annual cancer cases worldwide, making it among the most common cancer types in the world [https://www.breastcancer.org/facts-statistics, visited Apr. 11, 2024]. In 2023, an estimated 297,790 new cases of invasive breast cancer were expected to be diagnosed in U.S. women, along with 55,720 new cases of ductal carcinoma in situ (DCIS), a condition that affects the cells of the milk ducts in the breast.

In breast cancer, stage is based on the size and location of the primary tumor, the spread of cancer to nearby lymph nodes or other parts of the body, tumor grade, and whether certain biomarkers are present.

The American Joint Committee on Cancer (AJCC) provides two principal groups for breast cancer staging, as shown in Table 5. The first is anatomic, which is based on the extent of cancer as defined by tumor size (T), lymph node status (N), and distant metastasis (M). The second principal group is prognostic, which includes anatomic TNM plus tumor grade and the status of the biomarkers human epidermal growth factor receptor 2 (HER2), estrogen receptor (ER) and progesterone receptor (PR). Ductal carcinoma in situ and stage 1, stage IIA, stage IIB and stage IIIA breast cancers are considered early-stage breast cancer (i.e., cancer that has not spread beyond the breast or the axillary lymph nodes.

Prognostic stages are divided into clinical and pathological groups. The term “pathological stage applies to patients who have undergone surgery as the initial treatment for breast cancer. It includes all information used for clinical staging plus findings at surgery and pathological findings from surgical resection. Pathological prognostic stage does not apply to patients who received neoadjuvant therapy (e.g., systemic agents or radiation prior to surgical resection).

TABLE 5
AJCC Breast Cancer Staging
Primary Tumor (T)

| | |
|---|---|
| TX | Primary tumor cannot be assessed |
| T0 | No evidence of primary tumor |
| Tos | Carcinoma in situ |
| Tis(DIS) | Ductal carcinoma in situ |
| Tis(Paget) | Paget disease of the nipple NOT associated with invasive carcinoma |
| | and/or carcinoma in situ (DIS) in the underlying breast parenchyma. |
| | Carcinomas in the breast parenchyma associated with Paget disease are |
| | categorized on the basis of the size and characteristics of the |
| | parenchymal disease, although the presence of Paget disease should still |
| | be noted. |
| T1 | Tumors ≤20 mm in greatest dimension |
| T1mi | Tumors ≤1 mm in greatest dimension |
| T1a | Tumor >1 mm but ≤5 mm in greatest dimension (round any |
| | measurement >1.0-1.9 mm to 2 mm) |
| T1b | Tumor >5 mm but ≤10 mm in greatest dimension |
| T1c | Tumor >10 mm but ≤20 mm in greatest dimension |
| T2 | Tumor >20 mm but ≤50 mm in greatest dimension |
| T3 | Tumor >50 mm in greatest dimension |
| T4 | Tumor of any size with direct extension to the chest wall and/or to the |
| | skin (ulceration or skin nodules), not including invasion of dermis alone |
| T4a | Extension to chest wall, not including only pectoralis muscle |
| | adherence/invasion |
| T4b | Ulceration and/or ipsilateral satellite nodules and/or edema (including |
| | peau d'orange) of the skin, which do not meet the criteria for |
| | inflammatory carcinoma |
| T4c | Both T4a and T4b |
| T4d | Inflammatory carcinoma |

Regional lymph nodes (N)

| | |
|---|---|
| Clinical | |
| cNX | Regional lymph nodes cannot be assessed (e.g., previously removed) |
| cN0 | No regional lymph node metastasis (on imaging or clinical examination) |
| cN1 | Metastasis to movable ipsilateral level I, II axillary lymph node(s) |
| cN1mi | Micrometastases (approximately 200 cells, larger than 0.2 mm, but none |
| | larger than 2.0 mm |
| cN2 | Metastases in ipsilateral level I, II axillary lymph nodes that are |
| | clinically fixed or matted, or in ipsilateral internal mammary nodes in |
| | the absence of clinically evident axillary lymph node metastases |
| cN2a | Metastases in ipsilateral level I, II axillary lymph nodes fixed to one |
| | another (matted) or to other structures |
| cN2b | Metastases only in ipsilateral internal mammary nodes and in the |
| | absence of axillary lymph node metastases |
| cN3 | Metastases in ipsilateral infraclavicular (level III axillary) lymph |
| | node(s), with or without level I, II axillary node involvement, or in |
| | ipsilateral internal mammary lymph node(s) with level I, II axillary |
| | lymph node metastasis; or metastases in ipsilateral supraclavicular |
| | lymph node(s), with or without axillary or internal mammary lymph |
| | node involvement |
| cN3a | Metastasis in ipsilateral infraclavicular lymph node(s) |
| cN3b | Metastasis in ipsilateral internal mammary lymph node(s) and axillary |
| | lymph node(s) |
| cN3c | Metastasis in ipsilateral supraclavicular lymph node(s) |

Note: (sn) and (f) suffixes should be added to the N category to denote confirmation of
metastasis by sentinel node biopsy or fine needle aspiration/core needle biopsy, respectively

| | |
|---|---|
| Pathologic (pN) | |
| pNX | Regional lymph nodes cannot be assessed (for example, previously |
| | removed, or not removed for pathologic study) |
| pN0 | No regional lymph node metastasis identified histologically, or isolated |
| | tumor clusters (ITCs) only. Note: ITCs are defined as small clusters of |
| | cells ≤0.2 mm, or single tumor cells, or a cluster of <200 cells in a |
| | single histologic cross-section; ITCs may be detected by routine |
| | histology or by immunohistochemical (IHC) methods; nodes containing |
| | only ITCs are excluded from the total positive node count for purposes |
| | of N classification but should be included in the total number of nodes |
| | evaluated |
| pN0(i) | No regional lymph node metastases histologically, negative IHC |
| pN0(i+) | ITCs only in regional lymph node(s) |
| pN0(mol−) | No regional lymph node metastases histologically, negative molecular |
| | findings (reverse transcriptase polymerase chain reaction (RT-PCR) |
| pN0(mol+) | Positive molecular findings by RT-PCR; no ITCs detected |
| pN1 | Micrometastases; or metastases in 1-3 axillary lymph nodes and/or in |
| | internal mammary nodes; and/or in clinically negative internal |
| | mammary nodes with micrometastases or macrometastases by sentinel |
| | lymph node biopsy |
| pN1mi | Micrometastases (200 cells, >0.2 mm but none >2.0 mm) |
| pN1a | Metastases in 1-3 axillary lymph nodes (at least 1 metastasis >2.0 mm) |
| pN1b | Metastases in ipsilateral internal mammary lymph nodes, excluding |
| | ITCs, detected by sentinel lymph node biopsy |
| pN1c | Metastases in 1-3 axillary lymph nodes and in internal mammary |
| | sentinel nodes (i.e., pN1a and pN1b combined) |
| pN2 | Metastases in 4-9 axillary lymph nodes; or positive ipsilateral internal |
| | mammary lymph nodes by imaging in the absence of axillary lymph |
| | node metastases |
| pN2a | Metastases in 4-9 axillary lymph nodes (at least 1 tumor deposit >2.0 |
| | mm) |
| pN2b | Clinically detected* metastases in internal mammary lymph nodes with |
| | or without microscopic confirmation; with pathologically negative |
| | axillary lymph nodes |
| pN3 | Metastases in ≥10 axillary lymph nodes, or in infraclavicular (level III |
| | axillary) lymph nodes; or positive ipsilateral internal mammary lymph |
| | nodes by imaging in the presence of one or more positive level I, II |
| | axillary lymph nodes; or in >3 axillary lymph nodes and |
| | micrometastases or macrometastases by sentinel lymph node biopsy in |
| | clinically negative ipsilateral internal mammary lymph nodes; or in |
| | ipsilateral supraclavicular lymph nodes |
| pN3a | Metastases in ≥10 axillary lymph nodes (at least 1 tumor deposit >2.0 |
| | mm), or metastases to the infraclavicular (level III axillary lymph) nodes |
| pN3b | pN1a or pN2a in the presence of cN2b (positive internal mammary |
| | nodes by imaging) or pN2a in the presence of pN1b |
| pN3c | Metastases in ipsilateral supraclavicular lymph nodes |

*Clinically detected is defined as detected by imaging studies (excluding lymphoscintigraphy)
or by clinical examination and having characteristics highly suspicious for malignancy or a
presumed pathologic macrometastasis on the basis of fine needle aspiration (FNA) biopsy with
cytologic examination

Distant metastasis (M)

| | |
|---|---|
| M0 | No clinical or radiographic evidence of distant metastasis |
| cM0(i+) | No clinical or radiographic evidence of distant metastases in the |
| | presence of tumor cells or deposits no larger than 0.2 mm detected |
| | microscopically or by molecular techniques in circulating blood, bone |
| | marrow, or other non-regional nodal tissue in a patient without |
| | symptoms or signs of metastases |
| cM1 | Distant metastases detected by clinical and radiographic means |
| pM1 | Any histologically proven metastases in distant organs; or if in non- |
| | regional nodes, metastases >0.2 mm |

Table 6 below shows anatomic stage/prognostic groups
based on the AJCC TNM classification in Table 5.

| Stage | T | N | M |
|---|---|---|---|
| 0 | Tis | N0 | M0 |
| 1A | T1 | N0 | M0 |
| 1B | T0 | N1mi | M0 |
| | T1 | N1mi | M0 |
| IIA | T0 | N1 | M0 |
| | T1 | N1 | M0 |
| | T2 | N0 | M0 |
| IIB | T2 | N1 | M0 |
| | T3 | N0 | M0 |
| IIIA | T0 | N2 | M0 |
| | T1 | N2 | M0 |
| | T2 | N2 | M0 |
| | T3 | N1 | M0 |
| | T3 | N2 | M0 |
| IIIB | T4 | N0 | M0 |
| | T4 | N1 | M0 |
| | T4 | N2 | M0 |
| IIIC | Any T | N3 | M0 |
| IV | Any T | Any N | M1 |

Notes:
T1 includes T1mi.
T0 and T1 tumors with nodal micrometastases (N1mi) are staged as Stage 1B
T2, T3, and T4 tumors with nodal micrometastases (N1mi) are staged using the N1 category
M0 includes Mo(i+)
The designation pM0 is not valid; any M0 is clinical
If a patient presents with M1 disease prior to neoadjuvant systemic therapy, the stage is considered stage IV and remains stage IV regardless of response to neoadjuvant therapy.
Stage designation may be changed if post-surgical imaging studies reveal the presence of distant metastases, provided the studies are performed within 4 months of diagnosis in the absence of disease progression and provided the patient has not received neoadjuvant therapy.
Staging following neoadjuvant therapy is designated with “yc” or “yp” prefix to the T and N classification. No anatomic stage group is assigned if there is a complete pathologic response (pCR) to neoadjuvant therapy, for example, ypT0ypN0cM0.

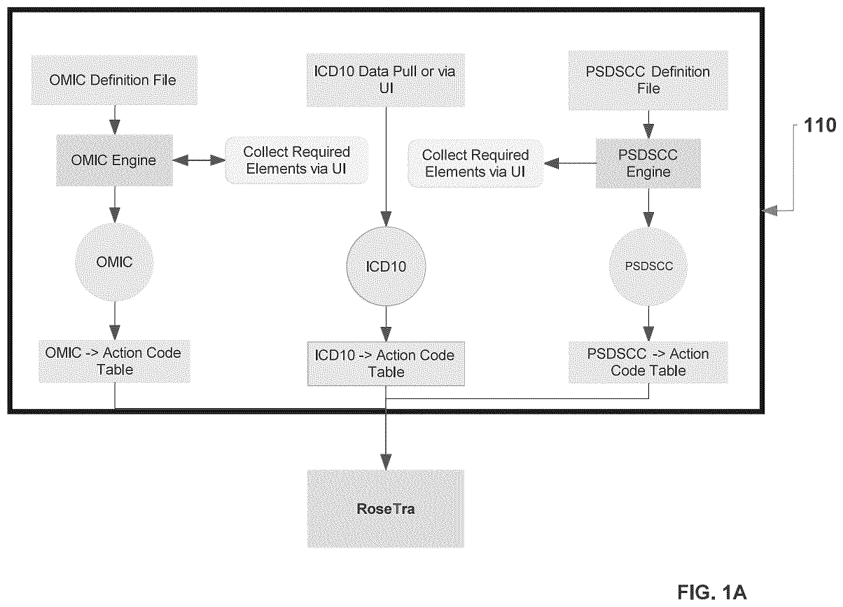

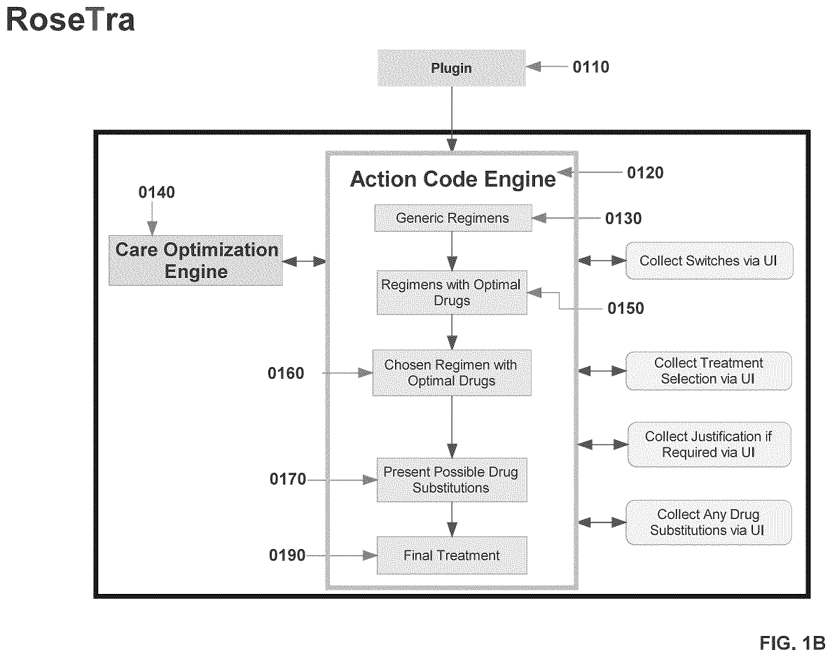

A block diagram depicting the inputs and outputs of the method in real time at point of care is shown in FIG. 1A and FIG. 1B.

FIG. 1A is a block diagram depicting exemplary plugins for a method 100 for identifying and presenting at a point of care a cost-optimized evidence-based path of care. The method comprises accessing, via input disease elements, a plug-in comprising a computer interface that accepts a patient disease-specific characterization code (PSDSCC) (110).

In some embodiments, the patient specific, disease-specific characterization code (PSDSCC) comprises a then current, new or updated collection of attributes or elements comprising values of predetermined variables that codifies information comprising the patient's specific disease type to a reduced number of clinically meaningful permutations

In some embodiments, the PSDSCC represents variables preselected by users to provide a set of clinically relevant patients. In some embodiments, the variables of the patient-specific disease-specific characterization code are selected by experts in the field.

In some embodiments, the PSDSCC is an ICD code. In some embodiments, the patient-specific disease-specific characterization code is a derived expression comprising a punctuated string of digits. In some embodiments, the patient-specific disease-specific characterization code is a derived expression comprising a punctuated strong of letters and symbols (OMIC). In some embodiments, the patient-specific disease-specific characterization code is a derived expression comprising an avatar (meaning a pictorial and symbol-based expression).

In some embodiments, the PSDSCC is an expression derived from alphabet letters, symbols and/or numbers of a common language, including one or more of Greek, Cyrillic and/or Latin character(s) that represent patient-specific parameters.

Exemplary parameters include, without limitation, at least one, some, or all of the following:
a racial classification of the patient (for example, Asian, Black, Hispanic, or Caucasian) and/or
disease type (for example, histology adenocarcinoma of lung), and/or stage classification in roman numerals (e.g., Stage I vs Stage II), and/or
a Greek, Cyrillic, or Latin letter and/or symbol (for example, Alpha (α); Beta (β)), and/or
a numeric symbol (+ or − or positive or negative) and/or
a number, and/or letter(s) and number(s) (i.e. KI67-30%), and/or
an expression of presence or absence, and/or
organ status (for example, heart failure), and/or
physiology status (premenopausal vs post-menopausal); and/or
age; and/or
sex; and/or
genomic type (and/or alteration); and/or
epigenetic type (and/or alteration); and/or
post-translational RNA type (and/or alteration); and/or
protein type; and/or
variant or alteration.

In some embodiments, the PSDSCC is an expression derived by machine learning. In some such embodiments, the patient-specific disease specific characterization code is an expression derived by deep learning.

In some embodiments, the PSDSCC is a derived expression comprising biometric patterns. In some embodiments, the patient-specific disease-specific characterization code is an expression comprising biometric patterns derived by machine learning. In some such embodiments, the patient-specific disease specific characterization code is an expression comprising biometric patterns derived by deep learning.

In some embodiments, the PSDSCC is a derived expression comprising a literal written description or a voice-activated description.

In some embodiments, the PSDSCC is a derived expression comprising sound patterns. In some embodiments, the patient-specific disease-specific characterization code comprising musical notes or tones. In some embodiments, the patient-specific disease-specific characterization code is an expression comprising sound patterns derived by machine learning. In some such embodiments, the patient-specific disease specific characterization code is an expression comprising sound patterns derived by deep learning.

In some embodiments, the PSDSCC is a derived expression comprising electromagnetic wave patterns. In some embodiments, the patient-specific disease-specific characterization code is a derived expression comprising light patterns including, without limitation, for example, nonconvergent light (e.g., visible light), convergent light (e.g., lasers), photons of light, or a combination thereof. In some embodiments, the patient-specific disease-specific characterization code is an expression comprising light patterns derived by machine learning. In some such embodiments, the patient-specific disease specific characterization code is an expression comprising light patterns derived by deep learning.

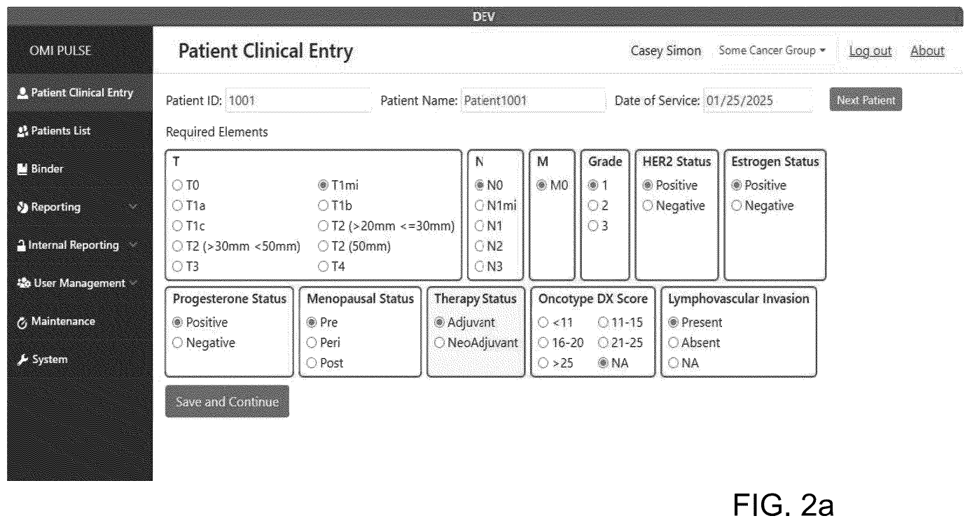

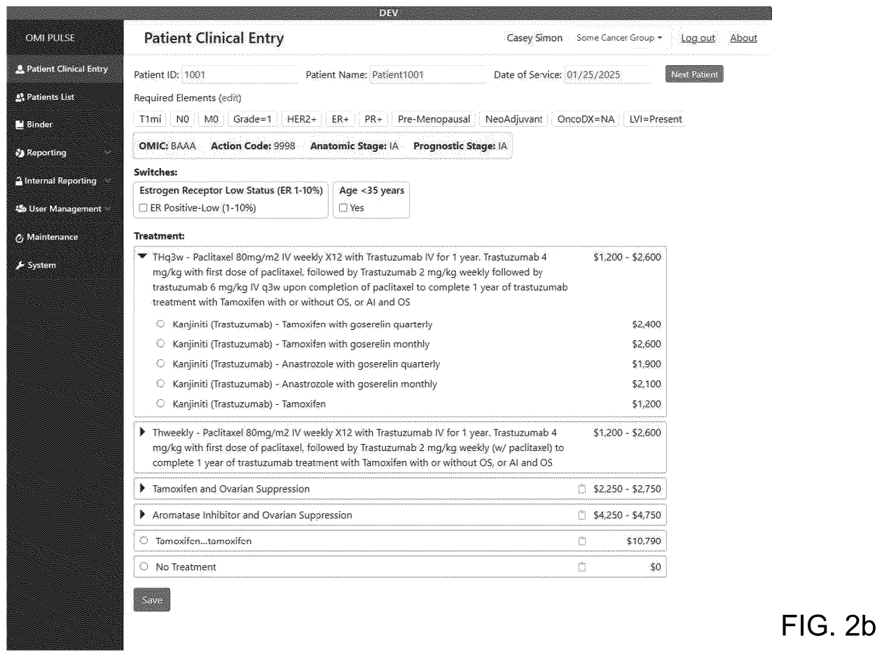

FIG. 2A and FIG. 2B are screenshots showing a graphical user interface (GUI) for transmitting and displaying information in accordance with inputs for an embodiment for a patient with breast cancer. For example, for breast cancer, exemplary attributes may include, without limitation, age, ethnicity, race, sex, ICD10 code, biomarker expression (e.g., hormone receptor (HR), estrogen receptor (ER), progesterone receptor (PR), human epidermal growth factor receptor 2 (HER2)); histologic grade, extent of lymphovascular invasion, menopausal status, tumor size; nodal involvement; metastatic sites, genomic biomarkers [e.g., breast cancer susceptibility genes BRCA1 and BRCA2; BRCA status; PALB2 status], ECOG at presentation, Oncotype DX assay; medical comorbidities; histology; therapy (surgery, adjuvant therapy; neoadjuvant therapy, radiation, etc.); and progression track.

Summary comorbidity measures, for example, the Charlson Index, Adult Comorbidity Evaluation 27 (ACE-27), attempt to assess the combined impact of different diseases. [See Geraci, J M et al, J. Clin. Oncol. (2005) 23(30): 7399-7404, which is incorporated by reference herein in its entirety].

The element ECOG performance status/quality of life metrics refers to a method by which the quality of life of the patient over time can be tracked. It is part of the demographic parameter disease specific clinical molecular phenotype, i.e., the stage of a patient's health at the start of therapy and is within Sorting. For example, a comparison of ECOG at start of therapy (e.g., ECOG of 3), with ECOG after therapy (e.g., ECOG of 2) reflects the effect of the therapy.

FIG. 1B is a block diagram depicting steps of the method whereby the patient disease-specific characterization code is translated by an action code engine [0120] into an action code, which collects a set of associated switches via a user interface (UI). The result of this collecting is a set of clinically equivalent candidate generic treatment regimens [0130], wherein drugs are listed by class. The generic regimens and action code engine communicate with a care-optimization engine to create regimens with optimal drugs [0150]. Treatment selections are selected via a UI to generate a chosen regimen with optimal drugs [0160]. Possible substitutions with equivalent drugs [0170] are collected from a healthcare provider via a UI. A treatment other than those indicated may be selected. In this case, a justification is required in order for the patient to remain in a value-based care program. The possible justifications are represented by “Justification Switches” and include reasons, such as ECOG Performance Status ≥3, Significant Comorbidity, Adverse Social Determinants of Health, and Patient Choice [180]. The output result is a final actual treatment regimen comprising a most cost-effective treatment plan.

In step (1) of the method, treatment choices based on evidence-based paths of care deemed appropriate for the patient are determined.

The action code engine [0120] generates, from values of the action code and a collected set of associated switches, a set of clinically equivalent candidate generic treatment regimens [0130] for patients that exhibit the disease specific characterization elements presented, wherein drugs are listed by class, i.e., no specific drugs are identified at this stage.

In the case of cancer, the generated set of clinically equivalent candidate generic treatment regimens [0130] comprises interchangeable NCCN-approved regimens for patients that exhibit the disease-specific characterization elements presented and other patient specific attributes, such as comorbidities, adverse social determinants of health, somatic genotypes that confer an increase in disease incidence, and/or delayed or accelerated drug metabolism, wherein drugs are identified only by drug class, i.e., no specific drugs are identified at this stage.

In some embodiments, the switches comprise optional (e.g., true/false, positive/negative; present/absent) elements. The switches codify specific health issues possessed by the patient, such that the value of the switches determines the set of treatment choices to be presented.

In some embodiments, there are a plurality of switches organized into groups or series—e.g., series I switches (1-99), series II switches (100-999), series III switches (1000+), etc, depending on the disease.

Exemplary series 1 switches for cancer (the 1-99 series) comprise disease modalities, including, without limitation, test results, e.g., ECOG, comorbidity; social determinants of health; patient choice; lymphovascular invasion; Oncotype DX DCIS; KI67; risk of breast cancer (breast cancer index); Mammaprint®; Endo Predict; Prosigna (PAM50); autoimmune disease/process; breast cancer subtypes; response to neoadjuvant therapy; PDL-1 expression; Estrogen Receptor (ER) status; HER-2 low; etc.

Exemplary Series II switches (the 100 to 999 series) for cancer include every known histology/histopathology of a tumor. Exemplary histopathology subtypes include, without limitation, acinar cell carcinoma; acinar cell cysadenocarcinoma; acinic cell carcinoma; adenocarcinoma-in situ-NOS (not otherwise specified); adenocarcinoma-in situ-mucinous, adenocarcinoma-in situ-non-mucinous; adenocarcinoma-minimally invasive mucinous; adenocarcinoma-minimally invasive non-mucinous; adenocarcinoma NOS (not otherwise specified); adenocarcinoma-acinar; adenocarcinoma-colloid; adenocarcinoma-cylindroid; adenocarcinoma-cribriform ductal; adenocarcinoma-ductal; adenocarcinoma-enteric; adenocarcinoma-fetal; adenocarcinoma-glandular dysplasia-intraepithelial neoplasia-high grade; adenocarcinoma-gastric faveolar type, etc.

Exemplary Series III switches (the 1000 series) for cancer include any genomic perturbation, including every gene that can be mutated, amplified, translocated, deleted, inserted, post-translationally modified, epigenetically altered (e.g., methylation or histone pattern regulating expression of the DNA) and/or at times, associated with aberrant messenger RNA, and any change in noncoding RNA is captured as a potential switch. Exemplary mutations include, without limitation, ABRAXAS1; APC, ATM; AKT1: Androgen Receptor-AR; BARD 1; BLM; BRCA1, BRCA2; BRPI1; CASP8, CTLA4, CYP19A1; CDH1; CHEK2; DIRAS 3; EGFR Exon 18 deletion-mutations E709 (delE709-T710insD); EGFR Exon 18 E709 substitution (X-A or G or K or V); EGFR Exon 18 G719X substitution (G7195 or G719A or G719C or G719D); EGFR Exon 19 deletion, etc.

The care optimization engine [0140] is where costs are calculated, and the most cost-efficient care is communicated to the main program and presented on a display screen. The care optimization engine optimizes every generic regimen choice and then orders them. The optimized regimens can be ordered e.g., by cost, by best reported Outcomes, or in other ways.

For example, the cumulative cost of each of the paths of care is determined by deriving a breakdown into a cost of care for every component of care (e.g., a formulation (pharmacy) and drug dispensing component, a supportive care component, a laboratory test component, an imaging component, a hospital visit component, a treatment component (including a surgery component, a radiation therapy component; a psychotherapy component; etc.) for each generic regimen for a one year period.

In step (2), the care-optimization engine optimizes the cost of the candidate generic treatment regimen choices by
(1) deriving a cost breakdown for each candidate generic treatment regimen over a one-year period and
(2) returning an actual treatment regimen comprising a most cost-effective treatment plan(s) comprising specific drugs substituted for the generic drug class.

The deriving a cost breakdown step at the point of care in real time can include, without limitation, at least the following examples:

Substituting drugs by identifying all primary medications and all choices for each of the primary medications that are biologically equivalent to select the lowest cost medications; for example, in one embodiment, for the drug component, treatment regimen 1 may include the Neulasta® brand of pegfilgrastim while treatment regimen 2 comprises the Udenyca® brand of pegfilgrastim. Both are the same, preapproved and equivalent agents.

Changing drug intervals (e.g., choice between giving a mediation once a week or once every three weeks);
Changing the pattern of care in real time such that it maintains or prevents the progression of disease severity so as to avoid unnecessary hospital days and ER visits;
Assessing heart function by the lowest cost effective method;
Reducing the number of ordered blood tests to eliminate those that are unnecessary;
Reducing the frequency of imaging (CAT scans, PET scans); Changing site of service from an expensive hospital base to a less expensive office space;
Using genomics to better identify and characterize disease so as to avoid medications that are not going to be effective and ultimately fail and to direct care to medications that will be effective upfront (e.g., melanoma where the patient has a BRAF mutation, and a course of chemotherapy will be ineffective);
Using genomic based diagnostic testing to better define when the disease process is truly eliminated or in a remission so medication can be stopped sooner.

For example, because the price of drugs changes every quarter, on a quarterly basis the cost optimization rules engine determines the lowest cost of care to enable optimization for average sales price while maximizing rebates, given the patient's fee schedule, which changes from insurance plan to insurance plan and how rebates are negotiated.

In step (3), the actual regimen comprising the evidence-based path of care associated with the best expected clinical outcome and the lowest total cost of care [0180]; and an ideal value-based path of care are presented to the healthcare provider on a display device at the point of care at a time of treatment decision.

In some embodiments, the presenting is through a graphic user interface (GUI).

In step [0170], the health-care provider is given an opportunity to substitute equivalent drugs for the ones determined as optimal for cost; although this will raise the cost, the provider has the opportunity to override the optimal cost selection for medical reasons. The healthcare provider, knowing the cost-optimized evidence-based path of care is then in the position to make an informed treatment decision.

In step 4, economic outcome data and clinical outcome data is displayed for monitoring and reporting over time. In some embodiments, the information is stored for later access by a user. In some embodiments, the information is transmitted to a user in response to an inquiry, request, or prompt directed to another computing system, another portion of the same computing system, or a remote system via a network. In some embodiments, the information enables the user (e.g., a healthcare provider (e.g., the physician or other appropriately licensed professional, e.g., a nurse), the practice or the healthcare system) to optimize drug utilization in accordance with specific purchasing contracts that include acquisition cost rebates and other economic benefits to enhance practice economic margins while reducing total cost of care and delivering an expected relevant clinical outcome [0180].

### Economic Outcomes

For example, in some embodiments, the method lowers the total cost of care and generates shared savings. In some embodiments, shared savings only occur for those patients with cancer where the optimal pathway is chosen and delivered dose intensity (DDI) is greater than or equal to 80%, i.e., 80%, 81%, 82%, 83%, 84%, 85%, 86%, 87%, 88%, 89%, 90%, 91%, 92%, 93%, 94%, 95%, 96%, 97%, 98%, 99%, or 100%.

In some embodiments, the method enables treatment of underserved patient populations with sub-adequate insurance by equalizing cost of acquisition of drug to reimbursement plus rebate.

In some embodiments, the method eliminates prior authorizations and precertification.

In some embodiments, the cost optimization process provides a basis for the negotiation of drug price by the healthcare provider's business side in order to optimize average sale price while maximizing rebates.

### Clinical Outcomes

For example, progression-free, disease-free survival and overall survival are monitored for any regimen selected in cancer care.

For example, delivered dose intensity (DDI) and Pathologic Complete Response (CR) are monitored for patients where a cancer care pathway is chosen.

In some embodiments, the method enables isolated healthcare providers to provide the recommended path of care, and at the lowest cost.

### Non-Transitory Computer-Readable Medium

According to another aspect, the present disclosure provides a non-transitory computer-readable medium storing instructions that, when executed by one or more processors of a computing system, performs the described method.

### System

According to another aspect, a system for facilitating identification of a cost-optimized evidence based path of care comprises storage; and one or more processors in communication with the storage and configured to execute instructions from the storage that, when executed by one or more processors of a computing system, cause the system upon an input disease element accessing a plug-in comprising an interface that accepts a patient specific disease-specific characterization code (PSDSCC), to:
(1) determine treatment choices based on evidence-based paths of care deemed appropriate for the patient by;
(a) translating by communication with an action code engine a patient specific disease-specific characterization code (PSDSCC) into an action code;
(b) collecting a set of action code-associated switches via the action code engine;
(c) generating from values of the action code and the set of associated switches, a set of clinically equivalent candidate generic treatment regimens comprising interchangeable regimens for patients that exhibit the disease-specific characterization elements presented, wherein drugs are identified only by drug class;
(2) optimize by communication with a care-optimization engine costs of all aspects of care that are changeable in the candidate generic regimen choices by
(a) identifying, characterizing, cost-assessing, and either avoiding or reducing its cost, and
(b) returning an actual treatment regimen comprising a most effective treatment plan;
(3) presenting by display on a display device at the point of care at a time of treatment decision by a user:
(i) the evidence-based actual treatment regimen, comprising the evidenced path of care associated with a best expected clinical outcome and a ranked total cost of care; and information regarding the paths of care; and
(ii) an ideal value-based path of care;
(4) collect, analyze and present by display economic outcome data and clinical outcome data for monitoring and reporting the data over time; and
(5) optimize drug utilization in accordance with specific purchasing contracts that include acquisition cost rebates and other economic benefits (for example, lower drug acquisition cost and lower administrative cost though removal of precertification and prior authorization) to enhance practice economic margins and reduce payor cost through reducing total cost of care and delivering an expected relevant clinical outcome.

In some embodiments, information regarding the cost optimized generic regimen paths of care includes all components of care, for example, pharmaceuticals and biologics, laboratory and imaging testing, hospitalization and emergency room use, surgery type, radiation therapy and vital organ function testing (for example echocardiogram).

In some embodiments, information to enable monitoring and reporting of economic outcomes for the patient includes data demonstrating lowering of the total cost of care and to generate shared savings compared to patients treated outside the system.

For example, in some embodiments, information may include data showing that shared savings may only occur for those patients with cancer where the optimal pathway is chosen and delivered dose intensity (DDI) is greater than or equal to 80%, i.e., 80%, 81%, 82%, 83%, 84%, 85%, 86%, 87%, 88%, 89%, 90%, 91%, 92%, 93%, 94%, 95%, 96%, 97%, 98%, 99%, or 100%.

For example, in some embodiments, the information may include data showing that the system enables treatment of underserved patient populations with sub-adequate insurance by equalizing cost of acquisition of drug to reimbursement plus rebate.

For example, in some embodiments, the information may include data showing that the system eliminates the need for prior authorizations and precertification in order to lower administrative burden and cost.

For example, in some embodiments, the information may include data showing that the system provides a basis for the negotiation of drug price by the healthcare provider's business side in order to optimize average sale price while maximizing rebates.

In some embodiments, information to enable monitoring and reporting of clinical outcomes for the patient compared to patients outside the system includes:

For example, in some embodiments, the information includes data obtained by monitoring progression-free survival, disease-free survival and overall survival for any regimen selected.

For example, in some embodiments, the information includes data obtained by monitoring delivered dose intensity (DDI) and Pathologic Complete Response (CR) are monitored for patients with cancer where a pathway is chosen.

For example, in some embodiments, the information includes data obtained by monitoring providing the recommended path of care, and at the lowest cost in geographic areas where isolated providers have limited alternatives.

### Computing Systems, Networks and Devices for Implementing Some Embodiments

The methods described herein can be implemented on one or more computing systems, networks or computing devices. Embodiments include systems for performing the methods described herein. Embodiments also include computer readable media holding instructions that, when executed, perform the methods described herein.

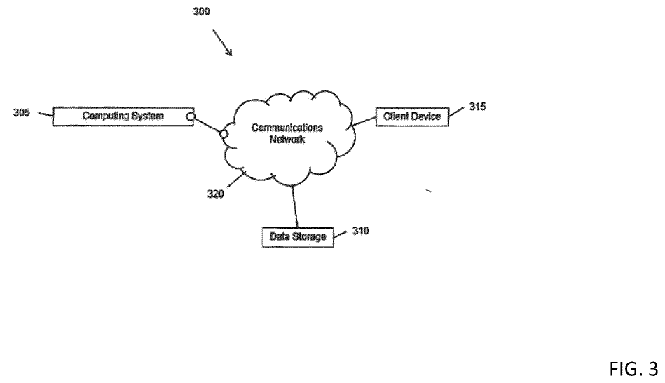

FIG. 3 schematically depicts a network 300, alternately described as a networked computing system, for implementing some aspects in accordance with some embodiments. Network 300 may include at least one computing system 305, at least one client device 315, and data storage 310 that may be in the form of one or more databases. In some embodiments, computing system 305, client device 315, readout device 317, and/or data storage 310 may be connected to network 320. However, in other embodiments, two or more of computing system 305, client device 315, and/or data storage 310 may be connected directly with each other, without network 320. While one computing system 305, one client device 315, and one data storage 310 are shown in FIG. 3A, it should be appreciated that any number of computing systems, client devices, and data storages could be used.

Computing system 305 may include one or more computing devices configured to perform one or more operations consistent with disclosed embodiments. Computing system 305 is further described in connection with FIG. 3A. In some embodiments, computing system 305 may perform at least some aspects or steps of the described methods. In some embodiments, computing system 305, and/or client device 315 may perform at least some aspects or steps of the described methods in some embodiments. For example, in some embodiments, client device 315 may be used to present a graphical user interface and may include an “app” for communication with a remote computing system.

Data storage 310 may include one or more computing devices configured with appropriate software to perform operations consistent with storing and providing data. Data storage 305 may include, for example, Oracle™ databases, Sybase™ databases, or other relational databases or non-relational databases, such as Hadoop™ sequence files, HBase™, or Cassandra™. Data storage 310 may include computing components (e.g., database management system, database server, etc.) configured to receive and process requests for data stored in memory devices of data storage 310 and to provide data from data storage 310. In some embodiments, data storage 305 may be configured to store current information from EMRs or an EMR system or any other data required by or produced by computing system 305 or client device 315. While data storage 310 is shown separately, in some embodiments, data storage 310 may be included in or otherwise related to computing system 305 and/or client device 315.

Client device 315 may include a desktop computer, a laptop, a server, a mobile device (e.g., tablet, smart phone, etc.), a wearable computing device, or other type of computing device. Client device 315 may include one or more processors configured to execute software instructions stored in memory, such as memory included in client device 315. In some embodiments, client device 315 may include software that when executed by a processor performs known Internet-related communication and content display processes. For instance, client device 315 may execute browser software that generates and displays interfaces including content on a display device included in, or connected to, client device 315. Client device 315 may execute applications that allows client device 315 to communicate with components over network 370 and generate and display content in interfaces via display devices included in client device 315. For example, client device 315 may display results produced by computing system 305, such as graphs, images etc. Computing system 305 may communicate results of analysis or reports to the client device 315.

Computing system 305, client device 315, and database 315 are shown as different components. However, computing system 305, client device 315, and/or database 315 may be implemented in the same computing system or device. For example, computing system 305, client device 315, and/or database 315 may be embodied in a single computing device. In some embodiments various functions or features could be implemented in a distributed manner or using a cloud computing or storage system instead of or along with computing system 305, data storage 310 and client device 315. In some embodiments, client device 315 may have an app installed that provides the GUI.

Network 320 may be any type of network configured to provide communications between components of network 320. For example, network 320 may be any type of network (including infrastructure) that provides communications, exchanges information, and/or facilitates the exchange of information, such as the Internet, a Local Area Network, near field communication (NFC), optical code scanner, or other suitable connection(s) that enables the sending and receiving of information between the components of network 320. In other embodiments, one or more components of network 320 may communicate directly through a dedicated communication link(s).

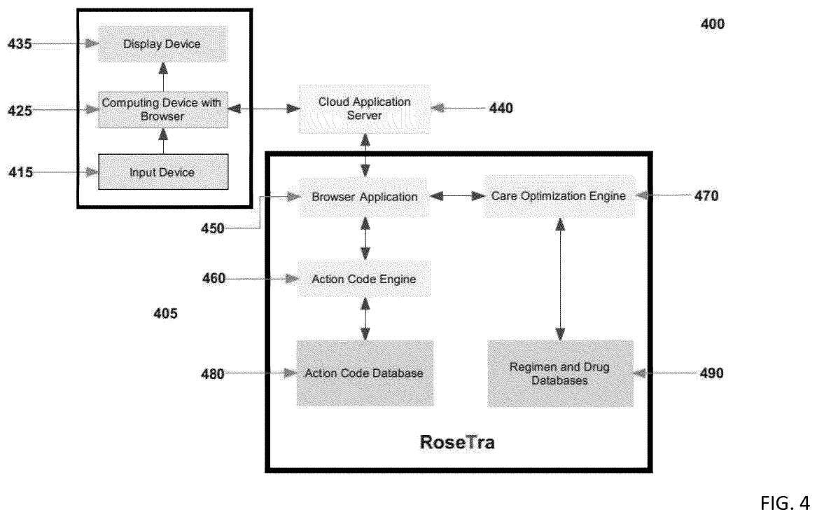

FIG. 4 is a block diagram schematically depicting one embodiment of a system of the present disclosure [0400]. In some embodiments, a client device comprises an input device (0415), a computing device with browser (0425) and a display device (0435). In some embodiments, the client device accesses the network architecture via a communication network (0440). In some embodiments, the communication network is a cloud application server. In some such embodiments, the cloud server interacts with a browser on a computer which, by accessing a website with permission, gains access to a program on the cloud server that interfaces with the system [0405].

In some embodiments, the system comprises three system components: a browser application [0450], an action code engine [0460], and a care-optimization engine [0470]. In some embodiments, the three system components are included in the same computing system. The browser application [0450], which runs the program, is communicatively linked to the action code engine [0460] and the care optimization engine [0490]. An action code database [0480] comprising switches interfaces with the action code engine [0460]. The action code database [0480] comprises, for every action code, the action code, text to be displayed for that action code, associated switches, and a list of the generic regimens that apply to that action code. A regimen and drug database [0490] interfaces with the care optimization engine. The regimen and drug database comprises all costs relating to treatment and the build/personalized treatment regimen, for each patient and regimen through one year of care comprising all aspects of each regimen. The care optimization engine processes the different details and combinations in the regimen and drug database. The final most cost-effective actual treatment regimen based on current prices is sent by the cost optimization engine to the browser which sends it to the action code engine.

In some embodiments, the system further includes an additional database comprising data storage [0495].

Although each of the browser, action code engine and care optimization engine is shown as distinct modules, it should be understood that they may be implemented as fewer or more modules than illustrated. It should be understood that any of the modules may communicate with one or more external components such as databases, servers, database servers or other devices.

In some embodiments, the browser application 450 is a software-implemented module, or a module implemented in part in software and in part in hardware, and is configured to accept a plugin comprising a patient specific disease-specific characterization code (PSDSCC) using a first machine learning-based model operating on values of a first subset of the plurality of elements/variables.

In some embodiments, the action code engine 460 is a software-implemented module, or a module implemented in part in software and in part in hardware and is configured to determine or generate an action code using a second machine learning-based model operating on values of a second subset of the plurality of elements/variables derived from the docked patient-specific disease-specific classification code.

In some embodiments, the care-optimization engine 470 is a software-implemented module, or a module implemented in part in software and in part in hardware, and is configured to determine an optimized cost for each candidate evidenced based path of care using a fourth machine learning-based model operating on values of a fourth subset of the plurality of elements/variables derived from an electronic communication from one or more switches.

Certain embodiments are described herein as including a number of components, modules, or mechanisms. Modules may constitute either software modules (e.g., code embodied on a machine-readable medium or in a transmission signal) or hardware modules. A hardware module is a tangible unit capable of performing certain operations and may be configured or arranged in a certain manner. In example embodiments, one or more computer systems (e.g., a standalone, client or server computer system) or one or more hardware modules of a computer system (e.g., a processor or group of processors) may be configured by software (e.g., an application or application portion) as a hardware module that operates to perform certain operations as described herein.

In various embodiments, a hardware module may be implemented mechanically or electronically. For example, a hardware module may include dedicated circuitry or logic that is permanently configured (e.g., as a special-purpose processor, such as a field programmable gate array (FPGA), and application-specific integrated circuit (ASIC) or a Graphics Processing Unit (GPU) to perform certain operations. A hardware module may also include programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired) or temporarily configured (e.g., programmed) to operate in a certain manner and/or to perform certain operations described herein. Considering embodiments in which hardware modules are temporarily configured (e.g., programmed), each of the hardware modules need not be configured or instantiated at any one instance in time. For example, where the hardware modules include a general-purpose processor configured using software, the general-purpose processor may be configured as respective different hardware modules at different times. Software may accordingly configure a processor, for example, to constitute a particular hardware module at one instance in time and to constitute a different hardware module at a different instance in time.

Hardware modules can provide information to, and receive information from, other hardware modules. Accordingly, the described hardware modules may be regarded as being communicatively coupled. Where multiples of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures in which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, include processor-implemented modules.

Similarly, the methods described herein may be at least partially processor-implemented. For example, at least some of the operations of a method may be performed by one or processors or processor-implemented modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., APIs).

Example embodiments may be implemented in digital electronic circuitry, or in computer hardware, firmware, software, or in combinations of them. Example embodiments may be implemented using a computer program product, for example, a computer program tangibly embodied in an information carrier, for example, in a machine-readable medium for execution by, or to control the operation of, data processing apparatus, for example, a programmable processor, a computer, or multiple computers.

In come embodiments, computing system 400 includes one or more non-transitory computer-readable media for storing one or more computer-executable instructions or software for implementing exemplary embodiments. The non-transitory computer-readable media can include, but are not limited to, one or more types of hardware memory, non-transitory tangible media (for example, one or more magnetic storage disks, one or more optical disks, one or more USB flash drives), and the like. For example, memory included in the computing system can store computer-readable and computer-executable instructions or software for implementing exemplary embodiments. Computing device 400 also includes processor and associated core, and optionally, one or more additional processor(s) and associated core(s) (for example, in the case of computer systems having multiple processors/cores), for executing computer-readable and computer-executable instructions or software stored in the memory and other programs for controlling system hardware. The processor(s) can each be a single core processor or multiple core processor.

Memory can include a computer system memory or random-access memory, such as DRAM, SRAM, EDO RAM, and the like. Memory can include other types of memory as well, or combinations thereof. An individual can interact with the computing system 400 through a visual display device/graphical user interface (GUI), such as a touch screen display or computer monitor, which can display one or more user interfaces for displaying data to the individual. The visual display device can also display other aspects, elements and/or information or data associated with exemplary embodiments. The computing system 400 can include other input devices and I/O devices for receiving input from an individual, for example, a keyboard, a scanner, or another suitable multi-point touch interface, a pointing device (e.g., a pen, stylus, mouse, or trackpad). The keyboard and the pointing device can be coupled to the visual display device. The computing system 400 can include other suitable conventional I/O peripherals.

The computing system 400 can also include one or more storage devices 495, such as a hard-drive, CD-ROM, or other computer readable media, for storing data and computer-readable instructions and/or software that implements exemplary embodiments of the system as described herein, or portions thereof. An exemplary storage device can also store one or more databases for storing suitable information required to implement exemplary embodiments. The databases can be updated by an individual or automatically at a suitable time to add, delete or update data in the databases. An exemplary storage device can store datasets, software, and other data/information used to implement exemplary embodiments of the systems and methods described herein.

The computing system 400 can include a network interface configured to interface via one or more network devices 420 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, processing device area network (CAN), or some combination of any or all of the above. The network interface can include a built-in network adapter, network interface card, PCMCIA network card, card bus network adapter, wireless network adapter, USB network adapter, modem or another device suitable for interfacing the computing system 400 to a type of network capable of communication and performing the operations described herein. Moreover, the computing system 400 can be a computer system, such as a workstation, desktop computer, server, laptop, handheld computer, tablet computer (e.g., the iPad® tablet computer), mobile computing or communication device (e.g., the iPhone® communication device), or other form of computing or telecommunications device that is capable of communication and that has sufficient processor power and memory capacity to perform the operations described herein.

The computing system 400 can run an operating system, such as versions of the Microsoft® Windows® operating systems, the different releases of the Unix and Linux operating systems, a version of the MacOS® for Macintosh computers, an embedded operating system, a real-time operating system, an open source operating system, a proprietary operating system, an operating systems for mobile computing devices, or another operating system capable of running on the computing device and performing the operations described herein. In exemplary embodiments, the operating system can be run in native mode or emulated mode. In an exemplary embodiment, the operating system can be run on one or more cloud machine instances.

The present invention may be embodied within a system, a method, a computer program product or any combination thereof. The computer program product may include a computer readable storage medium or media having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. In some embodiments, the program lives in the cloud, the data is stored in the cloud, the data is processed in the cloud, and results, when presented, are presented in an application that runs in the browser.

Computer readable program instructions described herein can be downloaded to the respective computing/processing devices from a computer readable storage medium or to an external computer or external storage device via a network. The computer readable program instructions may execute entirely on the user's computer, partly on the user's computer, as a stand-alone software package, partly on the user's computer and partly on a remote computer or entirely on the remote computer or server. Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which includes one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described.

All publications mentioned herein are incorporated herein by reference to disclose and described the methods and/or materials in connection with which the publications are cited.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A method for identifying and presenting at a point of care a care-optimized evidence-based path of care for a patient with a disease, comprising:
(1) determining treatment choices based on evidence-based paths of care deemed appropriate for the patient by:
(a) accessing a plug-in comprising an interface that accepts a patient specific disease-specific characterization code (PSDSCC);
(b) by communication with an action code engine translating the PSDSCC into an action code;
(c) the action code collecting a set of action code-associated switches via a user interface, wherein the action code links to actions by specifying a set of associated switches;
(d) generating, from values of the action code and the set of associated switches, a set of clinically equivalent candidate generic treatment regimens comprising interchangeable regimens for patients that exhibit the disease-specific characterization elements presented, wherein drugs are identified only by class;
(2) optimizing, by communication with a care-optimization engine, costs of all aspects of care that are changeable in the candidate generic treatment regimens to care for a specific illness by:
(a) identifying, characterizing, cost-assessing, and either avoiding or reducing its cost, and
(b) returning an evidence-based actual treatment regimen comprising a most effective treatment plan;
(3) presenting on a display device at the point of care at a time of treatment decision by a user:
(i) an evidence-based actual treatment regimen comprising the evidenced path of care associated with a best expected clinical outcome and a ranked total cost of care; and
(ii) an ideal value-based path of care;
(4) collecting, analyzing and presenting by display economic outcome data and clinical outcome data for monitoring and reporting the data over time; and
(5) optimizing drug utilization in accordance with site-specific purchasing contracts that include economic benefits comprising acquisition cost rebates to enhance practice economic margins and reduce payor cost through reducing total cost of care and delivering an expected relevant clinical outcome.

2. The method of claim 1, wherein the economic benefits include lower drug acquisition cost and lower administrative cost.

3. The method of claim 2 wherein lower administrative cost occurs through removal of precertification and prior authorization.

4. The method of claim 1, wherein the method is performed in real-time.

5. The method of claim 1, wherein
a. the PSDSCC is a derived expression comprising elements collected via a user interface; or
b. the PSDSCC is a derived expression comprising a punctuated string comprising letters and symbols (OMIC.); or
c. the PSDSCC is an expression derived from alphabet letters, symbols and/or numbers of a common language and include Greek, Cyrillic and/or Latin alphabet, symbols, and/or numbers; or
d. the PSDSCC is an ICD code; or
e. the PSDSCC is a derived expression comprising an avatar; or
f. the PSDSCC is a derived expression comprising biometric patterns; or
g. the PSDSCC is a derived expression comprising sound patterns; or
h. the PSDSCC is a derived expression comprising electromagnetic wave patterns.

6. The method according to claim 5, wherein
a. the electromagnetic wave patterns comprise patterns of light; and
b. the patterns of light include patterns of nonconvergent visible light, patterns of convergent laser light, patterns of photons of light, or a combination thereof.

7. The method according to claim 5 or claim 6, wherein the PSDSCC is an expression derived by machine learning or by deep learning.

8. The method of claim 1, wherein
a. each PSDSCC is associated with one action code, but each action code comprises a plurality of PSDSCCs; and
b. the action code resolves a choice of values of a plurality of disease elements.

9. The method of claim 1, wherein the associated switches specified by each action code comprise optional elements valued as true/false, positive/negative or present/absent.

10. The method of claim 1, wherein the components of care in step (2) include a pharmacy formulation component; a drug dispensing component, a supportive care component; a laboratory test component, an imaging component; a hospital visit component; a treatment component, a surgery component, a radiation component; and a psychotherapy/counseling component.

11. The method of claim 1, wherein the presenting is by a graphical user interface (GUI).

12. The method of claim 1, wherein the economic outcome comprises one or more of:
a) lowering the total cost of care;
b) generating shared savings;
c) eliminating prior authorization and precertification; or
d) providing a basis for the negotiation of drug price by the healthcare provider's business side in order to optimize average sale price while maximizing rebates.

13. The method of claim 12, wherein the economic outcome comprises equalizing cost of acquisition of drug to reimbursement plus rebate.

14. The method of claim 1, wherein the clinical outcome is:
(1) monitoring progression-free, disease-free survival and overall survival for any treatment regimen selected; or
(2) monitoring delivered dose intensity (DDI) and Pathologic Complete Response (CR) for patients where a pathway is chosen by a provider; or
(3) a combination thereof.

15. The method of claim 1, wherein the disease is a cancer.

16. The method of claim 15, wherein the cancer is an AIDS-related cancer; a breast cancer, a digestive/gastrointestinal cancer; an endocrine and neuroendocrine cancer; an eye cancer; a genitourinary cancer; a germ cell cancer; a gynecologic cancer; a head and neck cancer; a hematologic/blood cancer; a musculoskeletal cancer; a neurologic cancer; a respiratory/thoracic cancer; a skin cancer; or an unknown primary cancer.

17. The method of claim 15, wherein the cancer is a bladder cancer, breast cancer, colon and rectal cancer; endometrial cancer; kidney cancer; leukemia; liver cancer; lung cancer; melanoma; non-Hodgkin lymphoma; pancreatic cancer; prostate cancer; or thyroid cancer.

18. The method of claim 15, wherein in step 1(c), the set of clinically equivalent candidate generic treatment regimens comprising interchangeable regimens comprises NCCN-approved regimens for patients that exhibit the disease-specific characterization elements presented.

19. The method of claim 15, wherein the economic outcome comprises establishing a rule for distribution of shared savings such that shared savings only occur for those patients with cancer where the optimal pathway is chosen and delivered dose intensity (DDI) is greater than or equal to 80%.

20. A non-transitory computer-readable medium storing instructions that, when executed by one or more processors of a computing system, perform the method of claim 1.

21. A system for facilitating identification and presentation at a point of care of a cost-optimized evidence based path of care for a patient with a disease comprises storage and one or more processors in communication with the storage and configured to execute instructions from the storage that, when executed by one or more processors of a computing system, cause the system upon an input disease element accessing a plug-in comprising an interface that accepts a patient-specific disease-specific characterization code (PSDSCC) to:
(1) determine treatment choices based on evidence-based paths of care deemed appropriate for the patient by:
(a) by communication with an action code engine,
(i) translate the PSDSCC into an action code, and
(ii) collect a set of action code-associated switches;
(b) generate from values of the action code and the set of associated switches a set of clinically equivalent candidate generic treatment regimens comprising interchangeable regimens for patients that exhibit the disease-specific characterization elements presented, wherein drugs are identified only by drug class;
(2) optimize, by communication with a care-optimization engine costs of all aspects of care in the generic treatment regimens that are changeable to care for a specific illness by
a) identifying, characterizing, cost-assessing, and either avoiding or reducing their cost, and
b) return an actual treatment regimen comprising a most effective treatment plan;
(3) present on a display device at the point of care at a time of treatment decision by a user:
(i) the evidence-based actual treatment regimen comprising the evidenced path of care associated with a best expected clinical outcome and a ranked total cost of care and information regarding the paths of care; and
(ii) an ideal value-based path of care;
(4) collect, analyze and present by display economic outcome data and clinical outcome data for monitoring and reporting of the data over time; and
(5) optimize drug utilization in accordance with site-specific purchasing contracts that include acquisition cost rebates and other economic benefits to enhance practice economic margins and reduce payor cost through reducing total cost of care and delivering an expected relevant clinical outcome.

22. The system of claim 21, wherein the other economic benefits include lower drug acquisition cost and lower administrative cost.

23. The system of claim 22, wherein lower administrative cost is through removal of precertification and prior authorization.

24. The system of claim 21, wherein the steps are performed in real-time.

25. The system of claim 21,
(a) wherein the PSDSCC is a derived expression comprising elements collected through a user interface; or
(b) wherein the PSDSCC is a derived expression comprising a punctuated string comprising letters and symbols (OMIC); or
(c) wherein the PSDSCC is an ICD code; or
(d) wherein the PSDSCC is a derived expression comprising an avatar; or
(e), wherein the PSDSCC is a derived expression comprising biometric patterns; or
(f) wherein the PSDSCC is a derived expression comprising sound patterns; or
(g) wherein the PSDSCC is a derived expression comprising electromagnetic wave patterns.

26. The system of claim 25, wherein
a. the electromagnetic wave patterns comprise patterns of light; and
b. the patterns of light include patterns of nonconvergent visible light, patterns of convergent laser light, patterns of photons of light, or a combination thereof.

27. The system of claim 25 or 26, wherein the PSDSCC is an expression derived by machine learning or by deep learning.

28. The system of claim 21, wherein
(i) each PSDSCC is associated with one action code, but each action code comprises a plurality of PSDSCCs; and
(ii) the action code resolves a choice of values of a plurality of disease elements.

29. The system of claim 21, wherein the associated switches specified by each action code comprise optional elements valued as true/false, positive/negative, or present/absent.

30. The system of claim 21, wherein the presenting by display is by a graphical user interface (GUI).

31. The system of claim 21, wherein the components of care in step (3) include a pharmacy formulation component; a drug dispensing component, a supportive care component; a laboratory test component, an imaging component; a hospital visit component; a treatment component, a surgery component, a radiation component; and a psychotherapy/counseling component.

32. The system of claim 21, wherein economic outcomes comprise one or more of:
a) lowering the total cost of care; or
b) generating shared savings; or
c) eliminating prior authorization; or
d) providing a basis for the negotiation of drug price by the healthcare provider's business side in order to optimize average sale price while maximizing rebates.

33. The system of claim 21, wherein the economic outcome comprises equalizing cost of acquisition of drug to reimbursement plus rebate.

34. The system of claim 21, wherein the clinical outcome is one or more of:
(i) monitoring progression-free, disease-free survival and overall survival for any treatment regimen selected; or
(ii) monitoring delivered dose intensity (DDI) and Pathologic Complete Response (CR) for patients where a pathway is chosen by a healthcare provider.

35. The system of claim 21, wherein the disease is a cancer.

36. The system of claim 35, wherein the cancer is an AIDS-related cancer; a breast cancer, a digestive/gastrointestinal cancer; an endocrine and neuroendocrine cancer; an eye cancer; a genitourinary cancer; a germ cell cancer; a gynecologic cancer; a head and neck cancer; a hematologic/blood cancer; a musculoskeletal cancer; a neurologic cancer; a respiratory/thoracic cancer; a skin cancer; or an unknown primary cancer.

37. The system of claim 35, wherein the cancer is a bladder cancer, breast cancer, colon and rectal cancer; endometrial cancer; kidney cancer; leukemia; liver cancer; lung cancer; melanoma; non-Hodgkin lymphoma; pancreatic cancer; prostate cancer; or thyroid cancer.

38. The method of claim 15, wherein in step 1(c), the set of clinically equivalent candidate generic treatment regimens comprising interchangeable regimens comprises NCCN-approved regimens for patients that exhibit the disease-specific characterization elements presented.

39. The system of claim 35, wherein the economic outcome comprises establishing a rule for distribution of shared savings such that shared savings only occur for those patients with cancer where the optimal pathway is chosen and delivered dose intensity (DDI) is greater than or equal to 80%.
